SEQUENCE LISTING (20 sequences)

SEQ ID NO: 1 - DNA, 10 bp, Artificial Sequence
  Description of sequence Synthetic polynucleotide
tttttttttt

SEQ ID NO: 2 - DNA, 15 bp, Artificial Sequence
  Synthetic
ccccccccccaaaaa

SEQ ID NO: 3 - DNA, 10 bp, Artificial Sequence
  Synthetic
cccccccccc

SEQ ID NO: 4 - DNA, 10 bp, Artificial Sequence
  Synthetic
ccaacacaac

SEQ ID NO: 5 - DNA, 10 bp, Artificial Sequence
  Synthetic
ccaaccaacc

SEQ ID NO: 6 - DNA, 2 bp, Artificial Sequence
  Synthetic
cc

SEQ ID NO: 7 - DNA, 5 bp, Artificial Sequence
  Synthetic
aaaaa

SEQ ID NO: 8 - DNA, 9 bp, Artificial Sequence
  Synthetic
ggaaggaaa

SEQ ID NO: 9 - DNA, 9 bp, Artificial Sequence
  Synthetic
gagagagaa

SEQ ID NO: 10 - DNA, 10 bp, Artificial Sequence
  Synthetic
gagagagaaa

SEQ ID NO: 11 - DNA, 10 bp, Artificial Sequence
  Synthetic
cccccccggt

SEQ ID NO: 12 - DNA, 9 bp, Artificial Sequence
  Synthetic
aaaggaagg

SEQ ID NO: 13 - DNA, 9 bp, Artificial Sequence
  Synthetic
aagagagag

SEQ ID NO: 14 - DNA, 10 bp, Artificial Sequence
  Synthetic
aaagagagag

SEQ ID NO: 15 - DNA, 4 bp, Artificial Sequence
  Synthetic
caaa

SEQ ID NO: 16 - DNA, 6 bp, Artificial Sequence
  Synthetic
caaaac

SEQ ID NO: 17 - DNA, 43 bp, Artificial sequence
  Aptamer sequence
taacacgacagacgttcggaggtcgaaccctgacagcgtgggc

SEQ ID NO: 18 - DNA, 43 bp, Artificial sequence
  Aptamer sequence
taaccaccccacctcgctcccgtgacactaatgctaattccaa

SEQ ID NO: 19 - DNA, 66 bp, Artificial sequence
  Aptamer sequence
tgcgtgtgtagtgtgtctgggctacatcgatgagctgcctagggtccctcttagggatttgggcgg

SEQ ID NO: 20 - DNA, 66 bp, Artificial sequence
  Aptamer sequence
tgcgtgtgtactgtgtctggtcccgtagctactagcgaactccctgcctcttagggatttgggcgg

(19) United States
(12) Patent Application Publication
(10) Pub. No.: US 2023/0324384 A1
(43) Pub. Date: Oct. 12, 2023

(54) BAT ASSAYS FOR IN VITRO DETERMINATION OF ALLERGIC REACTION

(71) Applicant: DOTS Technology Corp., Natick, MA (US)

(72) Inventor: Adi Gilboa-Geffen, Wayland, MA (US)

(21) Appl. No.: 18/022,591

(22) PCT Filed: Sep. 9, 2021
(86) PCT No.: PCT/US2021/049567

§ 371 Date: Feb. 22, 2023

Related U.S. Application Data
(60) Provisional application No. 63/076,026, filed on Sep. 9, 2020.
(60) Provisional application No. 63/219,007, filed on Jul. 7, 2021.

(51) Int. Cl. G01N 33/569; G01N 33/58; G01N 33/50; G01N 21/64

(52) U.S. Cl. CPC G01N 33/56972; G01N 33/582; G01N 33/5047; G01N 21/6428; G01N 2333/70596; G01N 2800/24; G01N 2800/52; G01N 2021/6439

(57) ABSTRACT

The present disclosure relates to aptamer mediated BAT for diagnosing and/or prognosing an individual's allergic reaction to an allergen.

### CROSS REFERENCE TO RELATED APPLICATIONS

The present application claims priority to U.S. Provisional Patent Application No. 63/076,026 filed Sep. 9, 2020; and U.S. Provisional Patent Application No. 63/219,007 filed Jul. 7, 2021; the contents of each of which are incorporated herein by reference in their entirety.

### REFERENCE TO THE SEQUENCE LISTING

The present application is being filed along with a Sequence Listing in electronic format. The Sequence Listing is provided as a file entitled 2066-1016PCT_SEQLST.txt, created on Sep. 9, 2021, which is 4,186 bytes in size. The information in the electronic format of the sequence listing is incorporated herein by reference in its entirety.

### FIELD OF THE DISCLOSURE

The present disclosure relates to in vitro basophil activation test (BAT) for diagnosing and prognosing an individual's allergic reaction to an allergen such as a food allergen. The present BAT uses nucleic acid ligands (i.e., aptamer ligands) to measure basophil activation upon allergen stimulation.

### BACKGROUND OF THE DISCLOSURE

Allergy, specifically food allergy, is a major public concern estimated to affect about 5% of the population. Food allergy can be severe or life-threatening and is a common cause of food related anaphylaxis.

Today allergic individuals are diagnosed based on history of an IgE-mediated allergic reaction to an allergen with skin prick test (SPT) and/or detection of serum specific IgE (sIgE). Although SPT and sIgE have high sensitivity, they have low specificity to diagnose food allergy. Sometimes, even when using specific IgE to allergen components, the results can be equivocal. Oral Food Challenges (OFCs) are often applied when results of SPT and/or sIgE are inconclusive. However, without clear history of allergic reactions, interpretation of the results from the SPT and sigE methods are challenging. Furthermore, without an oral food challenge, it is difficult for a person who has allergy to determine how she/he is allergic to an allergen and how susceptible she/he is to a life-threatening reaction. The severity of an individual's allergy is hard to determine if the individual is not tested for an OFC. However, OFCs carry the risk of exposing patients to severe or life-threatening reactions, leaving long-lasting impacts on patient mental health.

As there is no cure for food allergy, the current management protocol, for a person who is diagnosed to have allergic reactions, is complete avoidance and use of an epinephrine autoinjector as rescue treatment. However, complete avoidance is difficult and accidental allergic reactions are common. Some of these reactions can be life-threatening even at a minimal amount of the allergen. The stress of a life-threating reaction could negatively affect the life quality. Food allergies pose a significant burden on the affected patients and their families. If an allergic test could determine the personal allergen threshold of an allergic individual, it could help to define the stringency of allergen avoidance and improve his/her quality of life. It is therefore of crucial importance to make the correct diagnosis.

Among recent emerging diagnostic allergy tests, such as histamine-release assays, specific epitope binding, and mast cell activation tests (Bahri et al., Mast cell activation test in the diagnosis of allergic disease and anaphylaxis. J Allergy Clin Immunol. 2018; 142:485-496; Larsen et al., A comparative study on basophil activation test, histamine release assay, and passive sensitization histamine release assay in the diagnosis of peanut allergy. Allergy. 2017; 73:137-144; and Beyer et al., Measurement of peptide-specific IgE as an additional tool in identifying patients with clinical reactivity to peanuts. J Allergy Clin Immunol. 2003; 112:202-207), basophil activation test (BAT) shows great potential for clinical applications in diagnosing and prognosing an individual's allergic reactions to allergens. The BAT has shown to be more accurate than IgE sensitization tests and able to distinguish individuals that were clinically allergic from those who were tolerant albeit sensitized in various studies (Santos et al., Basophil activation test discriminates between allergy and tolerance in peanut-sensitized children, J Allergy Clin Immunol. 2014; 134(3):645-652).

Basophils and mast cells are the effector cells of anaphylaxis. Recent research reports have demonstrated a linear correlation of activated basophils with the severity of allergic reactions. For example, a recent study reported that BAT reproduces very closely the phenotype of peanut-sensitized patients in relation to allergy versus tolerance (Santos et al., Basophil activation test discriminates between allergy and tolerance in peanut-sensitized children. J Allergy Clin Immunol., 2014; 134:645-652). The successful discrimination between allergy and tolerance provides useful information of the reaction severity to peanut in an individual. In the BAT, the percentage of activated basophils is correlated with the severity of an allergic reaction (e.g., food allergic reaction). A person with severe reaction to an allergen can show greater basophil reactivity and will respond to lower doses of the allergen (Santos et al., Distinct parameters of the basophil activation test reflect the severity and threshold of allergic reactions to peanut, J Allergy Clin. Immunol., 2015; 135(1):179-186). In the large peanut allergy study reported by Santos et al (J Allergy Clin Immunol. 2014; 134(3):645-652), BAT was externally validated in a new independent population and showed 100% specificity. The high specificity of the positive BAT to peanut confirmed peanut allergy and dispensed oral food challenge (OFC). These observations are consistent with the assumption that higher percentages of activated basophils result in increased basophil degranulation and increased release of vasoactive mediators, leading to more severe symptoms.

Implementing the BAT into the current allergist practice would have a significant effect on allergy management, particularly for food allergy management. The understanding of the severity of an allergic reaction an individual has would not only improve his/her quality of life but could also minimize accidental exposure. The basophil sensitivity in response to a particular allergen can be used as biomarkers of severity and threshold of the allergic reaction to that particular allergen.

The current BAT is a flow cytometry-based assay to detect expression of biomarkers on the surface of activated basophils such as CD63 and CD203c. In most assays, BAT uses antibodies to label basophil activation biomarkers. It is extremely difficult and costly to implement such a test in the allergy clinic settings though its significant effect on food allergy management.

The present disclosure implements a nucleic acid-based platform in which aptamer ligands that specifically bind to basophil biomarkers are used to measure basophil activation upon allergen stimulation.

### BRIEF DESCRIPTION OF THE DRAWINGS

FIG. 1 demonstrates the steps of the present BAT assay. A illustrates the step of sample process in which the heparinized whole blood is diluted with a buffer. B illustrates the step of incubation of the processed blood sample with aptamer ligands; the diluted blood sample is divided into 3 pods and are exposed to no peanut protein (negative control) and 2 test pods (0.1 g and 1.0 g peanut protein, respectively). C illustrates an exemplary labeling process in which two fluorescently labeled aptamer ligands against CD63 and CD203c are used to identify basophils in the blood sample. D illustrates the step of wash and signal read; after incubation, each sample is introduced to a dedicated reaction chamber that includes two anchors, one for each of the aptamer ligands.

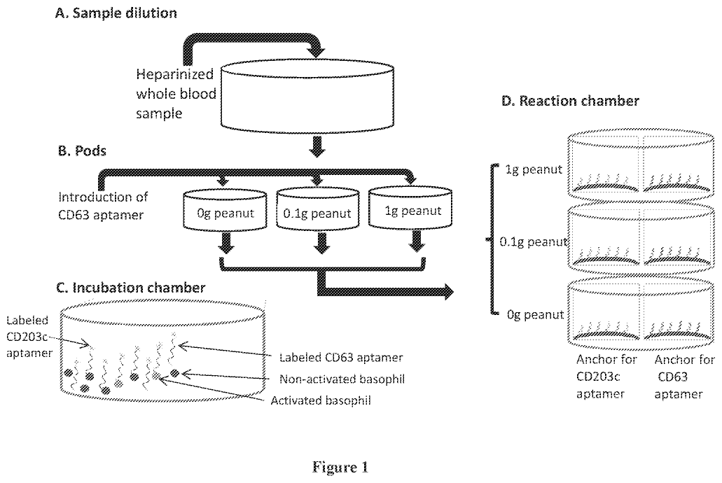

FIG. 2 shows the calculating the CD63/CD203c ratio in each peanut concentration (0.1 g and 1.0 g peanut protein) and comparison of the ratio between samples without peanut and samples containing peanut at different concentrations. Higher peanut concentration will have a signal closer to 1, while samples without peanut have a higher ratio. The upper panels (0 g peanut (A) and 1 g peanut (B)) show that non-activated basophils bind to CD203c. The baseline intensity is similar in all samples as CD203c aptamers that do not bind to basophils will bind to the anchors on the chip. The lower panel (A. 0 g peanut) shows that non-activated basophils do not bind to CD63. The baseline intensity is bright in the absence of allergen. CD63 aptamers that do not bind to basophils will bind to the anchors. The lower panel (B. 1 g aptamer) shows that activated basophils bind to CD63. The baseline intensity decreases in the presence of allergen. CD63 aptamers that do not bind to basophils will bind to the anchors.

FIG. 3A shows a representative embodiment of a detection system used to operate a BAT assay.

FIG. 3B shows the disposable pod demonstrating the reaction chamber inside the pod and the chip that is coated with anchors to capture signaling polynucleotides derived from aptamers.

FIG. 4 illustrates the assay as described in Example 2.

### SUMMARY OF THE DISCLOSURE

In one aspect of the present disclosure, basophil activation test (BAT) is provided in which nucleic acid ligands that specifically bind to biomarkers for basophils are used to identify basophils and measure the activation of basophils upon allergen stimulation, in a blood sample.

The BAT for measuring basophil activation in response to an allergen stimulation comprises identifying basophils in the blood sample using a nucleic acid ligand specifically binding to a biomarker of basophils; and detecting activated basophils with a nucleic acid ligand specific to a biomarker expressed on the cell surface of activated basophils. The ratio between total basophils and activated basophils as a function of allergen concentration.

In one embodiment, the BAT for measuring basophil activation in response to an allergen stimulation comprises identifying basophils in the blood sample using a nucleic acid ligand specifically binding to CD203c, a biomarker of basophils; and detecting activated basophils with a nucleic acid ligand specific to a biomarker expressed on the cell surface of activated basophils. The activation biomarker is CD63 and the nucleic acid ligand is an aptamer specifically binding to CD63.

The basophil activation induced by an allergen is measured by calculating the CD63/CD203c signal ratio.

In other embodiments, the BAT for measuring basophil activation in response to an allergen stimulation comprises identifying basophils in the blood sample using a nucleic acid ligand specifically binding to CD123, a biomarker of basophils; and detecting activated basophils with a nucleic acid ligand specific to the activation biomarker CD63. The basophil activation induced by an allergen is measured by calculating the CD63/CD123 signal ratio.

In another aspect of the present disclosure, a method for diagnosing and/or prognosing an individual's allergic reaction to an allergen of interest is provided; the method comprising measuring the activation of basophils after stimulating a blood sample from the individual with the allergen of interest. The method is referred to as basophil activation test (BAT). The basophil activation is measured by biomarkers expressed on the surface of activated basophils.

In some embodiments, the present BAT comprises the steps of: a) collecting a blood sample from the subject and incubating the blood sample with a test substance (e.g., an allergen of interest) to activate the basophils in the blood sample; b) introducing to the blood sample a mixture of nucleic acid ligands comprising an aptamer against an activation biomarker exposed on the cell surface upon activation of basophils, and an aptamer against an identification biomarker expressed on basophils, wherein each aptamer is labeled with a distinct fluorophore; c) capturing the nucleic acid ligands that are not bound to the basophil markers; d) reading the fluorescence signals and measuring the basophil activation; and e) calculating the basophil activation index by correlating the fluorescence signal changes and determining the allergic reaction.

In one exemplary embodiment, the basophil identification biomarker is CD203c, while the biomarker specific to activated basophil cells is CD63. An aptamer ligand specifically binding to CD203c is used to select the basophil population in the blood sample, while an aptamer ligand specifically binding to CD63 is used to label the activated basophils induced by the allergen exposure. The allergic reaction is therefore determined by the CD63/CD203c signal ratio.

In another exemplary embodiment, the basophil identification biomarker is CD123, while the biomarker specific to activated basophil cells is CD63. An aptamer ligand specifically binding to CD123 is used to select the basophil population in the blood sample, while an aptamer ligand specifically binding to CD63 is used to label the activated basophils induced by the allergen exposure. The allergic reaction is therefore determined by the CD63/CD123 signal ratio.

In some embodiments, the blood sample is processed to enrich basophil populations before stimulating the sample with the test substance.

In some embodiments, the labeled basophils in the blood sample are captured by flowing the sample over a chip coated with anchor sequences that are complementary to the aptamer ligands.

The present in vitro BAT is a functional test for determining the severity, threshold and degree of an individual's allergic reaction to an allergen of interest.

In some embodiments, the present in vitro BAT can be used to diagnose and/or diagnose an allergic reaction to a food allergen, such as peanut, soy, egg, wheat, milk, tree nuts, fish and shellfish. In other embodiments, the present in vitro BAT can be used to diagnose and/or diagnose the allergic reaction to an airborne allergen, an environmental allergen, a pathogen allergen, or a drug.

### DETAILED DESCRIPTION OF THE DISCLOSURE

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Unless defined otherwise, all technical and scientific terms used herein have the same meaning as commonly understood by one of ordinary skill in the art to which this disclosure belongs. In the case of conflict, the present description will control.

Food allergies are common and increasing in prevalence, representing a major health concern in many countries around the world. As complete prevention of allergy is not always possible, accurate diagnosis of an allergic reaction to an allergen is key in order to facilitate a targeted and safe management plan to prevent allergies. The current golden standard diagnosis of a supervised oral food challenges (OFCs) is not practically available or financially viable for all advances. An unmet need remains to develop accurate diagnostic means to clarify true diagnosis from false positivity and to categorize the severity and levels of allergic reaction to a specific allergen. A specific, sensitive, safe and rapid diagnostic method to determine a subject's allergic reaction upon exposure to a specific allergen is still desirable.

The basophil activation is emerging as a reliable and robust in vitro indicator of in vivo allergy reactions. Basophil activation test (BAT) to measure the basophil activation is a new diagnostic test which has high specificity and sensitivity and can complement specific igE (sIgE). The BAT has gained increasing interest for allergy diagnosis as an increasingly attractive in vitro diagnostic tool. However, the current BAT is a flow-cytometric based assay which is complicated techniques and proper equipment is required. Moreover, the current BAT uses antibodies specific to markers associated with basophils to measure the basophil activation. An improved and simple test would advance the current BAT for allergy diagnosis.

In accordance with the present disclosure, provided is an aptamer-based BAT for diagnosing and prognosing an individual's allergic reaction to an allergen of interest. Accordingly, the present in vitro BAT uses nucleic acid ligands (e.g., aptamer ligands) to identify and label activated basophils in responding to an allergen such as a food allergen. The nucleic acid ligands are aptamers selected by SELEX methods, which specifically bind to biomarkers associated with basophils. The aptamer ligands can be further modified to create signaling polynucleotides (SPNs) for labeling basophils.

Furthermore, instead of using flow cytometric techniques to capture basophils, a platform that implements hybridization of a nucleic acid aptamer ligand and its complementary anchor probes to capture basophils.

The current aptamer-based BAT can be implemented in connection with any suitable detection devices and systems. As non-limiting examples, the detection systems may include those disclosed by Applicant in PCT patent application publication Nos.: WO2016149253, WO2017/160616, WO2018/156535, and WO2019/165014, and PCT patent application No.: PCT/US2019/054599; the contents of each of which are incorporated herein by reference in their entirety.

In some embodiments, the detection system includes a sampler to collect the specimen, a single-use pod that contains all the chemicals needed to run the detection assay, and an instrument that runs and analyzes the assay.

The present in vitro BAT has a superior specificity and sensitivity to currently available tests. It can predict severity, and threshold to an allergen, and the likelihood of allergy persistence. Moreover, the present BAT can aid in identifying patients most suitable for immunotherapy.

### Definitions

Allergy: As used herein, terms “allergy” “allergic reaction,” and “allergic response” are used interchangeably to describe an abnormal immune reaction to an encountered allergen introduced by inhalation, ingestion or skin contact. The terms also refer to clinically adverse reactions to environmental allergens and drugs which reflect the expression of acquired immunologic responsiveness involving allergen-specific antibodies and/or T cells. These terms also include adverse immunologic responses that are associated with the production of allergen specific IgE.

Allergen: As used herein, the term “allergen” refers to any substance that induces an allergy in a susceptible subject. Allergens include any antigens that elicit a specific IgE associated immune responses. Common allergens include but are not limited to pollen, grasses, dust, as well as foods, including, but not limited to, nuts, milk, eggs, shellfish, and venoms, and various drugs. Allergens include, without limitation, nanoparticles, metal or metal alloys, drug or medicine related antigens; various biological matters, e.g., proteins, which may be related to animals such as insects or arachnids. As such, allergens are typically referred to as antigens.

Biomarker: As used herein, the term “biomarker” refers to molecules including, without limitation, proteins, nucleic acids, and metabolites, together with their polymorphisms, isoforms, mutations, derivatives, variants, modifications, and precursors, including nucleic acids and pro-proteins, cleavage products, receptors (including soluble and transmembrane receptors), subunits, fragments, ligands, protein-ligand complexes, multimeric complexes, and degradation products, elements, related metabolites, and other analytes or sample-derived measures. Biomarkers can also include mutated proteins or mutated nucleic acids. Biomarkers also include any calculated indices created mathematically or combinations of any one or more of the foregoing measurements. In the context of the present disclosure, a biomarker is a molecule specific to basophils which allow the correct identification of basophils.

Complementary: As used herein, the term “complementary” or “complement” refer to the natural binding of polynucleotides by base pairing such as A-T(U) and C-G pairs. Two single-stranded molecules may be partially complementary such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands.

Flow cytometry. As used herein, the term “flow cytometry” refers to a process in which physical and/or chemical characteristics of single cells, or by extension, of other biological or nonbiological particles in roughly the same size or stage, are measured. In flow cytometry, the measurements are made as the cells or particles pass through the measuring apparatus (flow cytometer) in a fluid stream. A cell sorter, or flow sorter, is a flow cytometer that uses electrical and/or mechanical means to divert and collect cells (or other small particles) with measured characteristics that fall within a user-selected range of values.

Hybridization: As used herein, the term “hybridization” refers to the process by which a polynucleotide strand anneals with a complementary strand through base pairing under defined hybridization conditions. Specific hybridization is an indication that two nucleic acid sequences share a high degree of identity such as an aptamer ligand of the present disclosure and a short complementary sequence of the aptamer. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched.

Ligand: As used herein, the term “ligand” refers to any molecule that can bind to a target such as a receptor and a biomarker on the cell surface, etc. A ligand includes, but is not limited to a protein, an antibody, a polypeptide, a nucleic acid (DNA, RNA, and modified forms thereof), an oligonucleotide (e.g., an aptamer), a peptide, a peptoid, a polyamine, a carbohydrate, a lipid and a small molecule. In the context of the present disclosure, a ligand is a nucleic acid molecule, particularly an aptamer that specifically binds to a biomarker, e.g., a biomarker for basophils.

Nucleic acid: As used herein, the terms “nucleic acid,” “polynucleotide,” “oligonucleotide” are used interchangeably. A nucleic acid molecule is a polymer of nucleotides consisting of at least two nucleotides covalently linked together. A nucleic acid molecule is a DNA (deoxyribonucleotide), an RNA (ribonucleotide), as well as a recombinant RNA and DNA molecule or an analogue of DNA or RNA generated using nucleotide analogues. The nucleic acids may be single stranded or double stranded, linear or circular. The term “nucleic acid” in its broadest sense, includes any compound and/or substance that comprise a polymer of nucleotides. These polymers are often referred to as polynucleotides. Exemplary nucleic acid molecules or polynucleotides of the disclosure include, but are not limited to, either D- or L-nucleic acids, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a. β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. The term also comprises fragments of nucleic acids, such as naturally occurring RNA or DNA which may be recovered using the extraction methods disclosed, or artificial DNA or RNA molecules that are artificially synthesized in vitro (i.e., synthetic polynucleotides). Molecular weights of nucleic acids are also not limited, may be optional in a range from several base pairs (bp) to several hundred base pairs, for example from about 2 nucleotides to about 1,0000 nucleotides, or from about 10 nucleotides to 5,000 nucleotides, or from about 10 nucleotides to about 1,000 nucleotides.

The term “nucleotide” refers to the monomer of nucleic acids, a chemical compound comprised of a heterocyclic base, a sugar and one or more phosphate groups. The base is a derivative of purine and pyrimidine and the sugar is a pentose, either deoxyribose or ribose.

Sample: As used herein, the term “sample” refers to any composition that might contain a target of interest to be analyzed including, but not limited to, biological samples obtained from subjects such as blood samples, samples obtained from the environment for example soil samples, water samples, agriculture samples (including plant and crop samples), or food samples. Food samples may be obtained from fresh food, processed/cooked food or frozen food.

Sensitivity: As used herein, the term “sensitivity” means the ability of a detection molecule to bind to a target molecule. The terms “sensitivity” and “reactivity” are used interchangeably.

Specifically bind: as used herein, the term “specifically bind” means that an aptamer reacts or associates more frequently, more rapidly, with greater duration and with greater affinity with a particular target molecule, than it does with alternative target molecules. For example, an aptamer that specifically binds to a target allergen binds that allergen or a structural part or fragment thereof with greater affinity, avidity, more readily, and/or with greater duration than it binds to unrelated allergen protein and/or parts or fragments thereof. It is also understood by reading this definition that, for example, an aptamer that specifically binds to a first target may or may not specifically bind to a second target. As such, “specific binding” does not necessarily require exclusive binding or non-detectable binding of another molecule, this is encompassed by the term “selective binding”. The specificity of binding is defined in terms of the comparative dissociation constants (Kd) of the aptamer for target as compared to the dissociation constant with respect to the aptamer and other materials in the environment or unrelated molecules in general. Typically, the Kd for the aptamer with respect to the target will be 2-fold, 5-fold, or 10-fold less than the Kd with respect to the target and the unrelated molecule or accompanying molecule in the environment. Even more preferably, the Kd will be 50-fold, 100-fold or 200-fold less.

Subject: As used herein, the terms “subject,” “individual” and “patient” are used interchangeably, and refer to a vertebrate, preferably a mammal, more preferably a human. Mammals include, but are not limited to, murine, simians, humans, farm animals, sport animals, and pets.

Target: as used herein, the term “target” and “target molecule” refers to a molecule which may be found in a tested sample, and which is capable of binding to a detection molecule such as an aptamer or an antibody.

### Basophil Activation Test (BAT)

Basophils are one class of leukocytes circulating in the blood stream and belong to the granulocytes. Despite their low abundance in human blood (less than 2% of the leukocyte fraction), basophils, like mast cells, are recognized as important effector cells in allergic hypersensitivity reactions by releasing potent inflammatory mediators including histamine and leukotrienes.

Immunoglobulin E (IgE) represents one of the classes of immunoglobulins. It is known to participate in allergic reactions. Circulating IgE molecules bind to the basophil membrane via the high affinity IgE receptor (FcεRI) on basophils. In this context, A new basophil assay using flow cytometry technique called basophil activation test (BAT) has gained increased interest for diagnosing allergic reactions to a variety of allergens. The BAT is a functional assay that measures IgE function, i.e., its ability to induce the activation of basophils in the presence of allergen. The in vitro BAT closely replicates type I hypersensitivity reactions, which develop in vivo when allergic individuals are exposed to the allergen, and thus can have clinical applications in the diagnosis and prognosis of allergic disease, alongside research applications.

The BAT measures the percentage of activated basophils after allergen stimulation by measuring expression of biomarkers on the surface of activated basophils that are upregulated following the cross-linking of IgE antibodies bound to the high-affinity igE receptor (FcεRI) resulting from allergen or anti-IgE stimulation. Several reports have demonstrated that the percentage of activated basophils is tightly correlated with the severity of an allergic reaction (e.g., food allergic reaction). An individual with severe reaction to an allergen can show greater basophil reactivity and responds to lower doses of the allergen (Alexandra et al., Distinct parameters of the basophil activation test reflect the severity and threshold of allergic reactions to peanut, J Allergy Clin Immunol, 2015; 135(1):179-186).

Basophil reactivity is closely relevant to allergen-induced IgE-mediated allergic reactions and anaphylaxis; the severer the allergic reaction, the greater basophil reactivity (i.e., the higher percentage of activated basophils). Higher percentages of activated basophils would result in higher percentages of basophils degranulating and higher amounts of vasoactive mediators released, leading to more severe symptoms. Santos et. al reported that allergen-specific basophil reactivity (as measured by CD63 peanut/anti-IgE) and basophil sensitivity (as measured by CD-sens) could be used as biomarkers of severity and threshold of allergic reactions to peanut during OFCs. Therefore, the BAT is useful for determining the severity, threshold and degree of an allergic reaction to a particular allergen such as a food allergen.

The precise identification of the population of basophils is a prerequisite for a valid interpretation of test results. A number of different markers can be used to identify basophils and to quantify their activation by flow cytometry. The cell-surface markers or their combinations can allow the correct identification of basophils.

As used herein, the term “identification biomarker” refers to a molecule that is constitutively and specifically expressed on a particular type of cell. For example, CD203c is constitutively and specifically expressed on basophil and is often used as an identification biomarker of basophils. In some embodiments, identification markers specific to basophils may be used in the BAT for positive selection of basophils from a sample. For example, markers that can identify basophils include, but are not limited to, CCR3, CD23c, CD123, CD3, IgE and CRTH2.

As used herein, the term “activation biomarker” refers to a molecule that is induced to expose to the cell surface upon allergen stimulation, thereby indicating basophil activation. Molecules associated tightly with basophil activation upon stimulation of a test substance (e.g., an allergen) may be used to measure the basophil activation. CD63 (a.k.a. lysosomal-associated membrane protein (LAMP-3)) has been discovered as the basophil activation marker (Knol et al., Monitoring human basophil activation via CD63 monoclonal antibody 435. J Allergy Clin Immunol. 1991: 88:328-338). CD63 is mainly associated with membranes of intracellular vesicles and can be induced to express on the cell surface of activated basophil cells, therefore serving as a biomarker for flow cytometric quantification of activated basophils in the BAT. The basophil activation markers include, but are not limited to CD203c, CD63, CD13, CD69, CD107a, CD107b, CD164, CD80, CD86, CD40L, HLA-DR, CD123, CD193, CRTH12, CCR3 and other extracellular markers on basophils, or intracellular markers such as Ph-CREB, Ph-STATS, Ph-S6rp, Ph-eIF4E, CREB or mTOR pathway proteins, or other phosphorylation related markers, or other proteins or small molecules related to the activation of basophils. Some basophilic phosphorylation of intracellular molecules, such as p38, MAPK and STAT5, can alternatively be used to measure basophil activation (Ebo et al., Combined analysis of intracellular signaling and immunophenotype of human peripheral blood basophils by flow cytometry: a proof of concept. Clin Exp Allergy. 2007; 37(11):1668-1675). The physiological condition may be the result of an exposure to a substance, allergen, drug, protein, chemical, or other stimulus, or maybe the result of removal of a substance, allergen, drug, protein, chemical or other stimulus.

As used herein, the term “cell surface marker” refers to an antigenic determinant or epitope found on the surface of a specific type of cell. Cell surface markers can facilitate the characterization of a cell type, its identification, and eventually its isolation. Cell sorting techniques are based on cellular biomarkers where one or more cell surface markers are used for either positive or negative selection, i.e., for inclusion or exclusion, from a cell population.

CD203c and CD63 are the most commonly used basophil identification and activation markers, respectively. CD203c is the only lineage-specific basophil marker that is constitutively and specifically expressed on basophils and often used as a single identification marker or in combination with other markers. CD63 is up regulated on the surface of basophils following stimulation with allergen and activation/degranulation of basophils. CD193 and CD123 can also be used as basophiil markers.

The application of BAT in food allergy was reported for various food allergens such as peanut (Sabato et al., Basophil activation reveals divergent patient-specific responses to thermally processed peanuts. J Investig Allergol Clin Immunol. 2011; 21(7):527-531) and milk (Rubio et al., Benefit of the basophil activation test in deciding when to reintroduce cow's milk in allergic children. Allergy. 2011; 66(1):92-100)). BAT shows a high specificity and sensitivity in many allergic reaction investigations. For example, in a large peanut allergy study, BAT was externally validated in a new independent population and showed 100% specificity. The high specificity means that a positive BAT to peanut confirmed peanut allergy and dispensed oral food challenge (OFC) (Santos et al., Basophil activation test discriminates between allergy and tolerance in peanut-sensitized children. J Allergy Clin Immunol 2014; 134(3):645-652). In Rubio's milk test, the BAT yielded the highest specificity (90%) and sensitivity (91%), the highest positive predictive value with 81%, and the highest negative predictive value with 96%, as compared with skin test, and anti-cow's milk IgE antibody measurement (Allergy. 2011; 66(1):92-100). Similar results were obtained with other foods, such as egg and tree nut allergens.

Antibodies against basophil identification and activation biomarkers are often used to label basophils in the BAT for flow cytometry sorting of labeled basophils. An improved BAT with simpler technique to measure basophil activation. The present application discloses a nucleic acid ligand-based BAT platform for food allergy diagnosis and/or prognosis. In one exemplary embodiment, the nucleic acid ligand is an aptamer ligand wherein the aptamer replaces antibodies against basophil cell markers used in current BAT assays and wherein the aptamer binds to the basophil cell markers with high affinity and specificity.

### Aptamers

In accordance with the present disclosure, a nucleic acid-based ligand that specifically binds to a biomarker is used. The nucleic acid ligand is an aptamer or a derivative thereof.

Aptamers (sometimes also called chemical antibodies) are single-stranded oligonucleotides (RNA or single stranded DNA) that form stable but unique three-dimensional confirmations capable of binding with high affinity and specificity to a variety of molecular targets. Aptamers bind to protein targets in much the same manner as antibodies and modulate protein function. Aptamers have advantages over antibodies in that they are poorly immunogenic, stable, and often bind to a target molecule more strongly than do antibodies. Generally, aptamers can be synthesized easily and in large quantities by in vitro transcription, PCR, or chemical synthesis (Annu. Rev. Med. 2005, 56, 555-583; Nat Rev. Drug Discov. 2006, 5, 123-132), and target-specific aptamers can be selected from random-sequence, single-stranded nucleic acid libraries by an in vitro selection and amplification procedure known as SELEX (systematic evolution of ligands by exponential enrichment). The selected aptamers are small single-stranded nucleic acids that fold into a well-defined three-dimensional structure. They show a high affinity and specificity for their target molecules. Furthermore, aptamers have important properties that simplify its industrialization. For example, aptamers are thermally stable, so they can be stored and transported easily. Aptamers can be produced or modified in large scale, with minimal batch-to-batch variation, given the well-established chemical synthesis and modification technologies.

Aptamers are useful and cost-effective tools for biochemical analyses. Also, they can be developed quickly against a seemingly unlimited range of targets. To date, specific aptamers against diverse targets have been successfully developed, including small inorganic irons, organic peptides, drugs, proteins (e.g., biomarkers), lipids and even complex cells and microorganisms. Recently several reviews outline aptamer applications in a variety of fields as targeting ligands including biomarkers of interest (Guan et al., Aptamers as versatile ligands for biomedical and pharmaceutical applications, Int. J Nanomedicine., 2020; 15:1059-1071; and Ozalp et al., Aptamers: molecular tools for medical diagnosis. Curr. Top Med Chem., 2015; 15(12):1125-1137; the contents of each of which are incorporated herein by reference in their entirety).

As used herein, an “aptamer” is a biomolecule that binds to a specific target molecule and modulates the target's activity, structure, or function. An aptamer of the present disclosure may be nucleic acid or amino acid based. Nucleic acid aptamers have specific binding affinity to molecules through interactions other than classic Watson-Crick base pairing. Nucleic acid aptamers, like peptides generated by phage display or monoclonal antibodies (mAbs), are capable of specifically binding to selected targets and, through binding, block their targets' ability to function. In some cases, aptamers may also be peptide aptamers. As used herein, an “aptamer” specifically refers to either a nucleic acid aptamer or peptide aptamer.

A typical nucleic acid aptamer is approximately 10-15 kDa in size (20-45 nucleotides), binds its target with sub-nanomolar affinity, and discriminates against closely related targets. Nucleic acid aptamers may be RNA (ribonucleic acid), DNA (deoxyribonucleic acid), or mixed DNA and RNA. Aptamers may be single stranded RNA, DNA or mixed RNA and DNA.

Aptamers may be either monovalent or multivalent. Aptamers may be monomeric, dimeric, trimeric, tetrameric or other higher multimeric. Individual aptamer monomers may be linked to form multimeric aptamer fusion molecules. As a non-limiting example, a linking oligonucleotide (i.e., linker) may be designed to contain sequences complementary to both 5′-arm and 3′-arm regions of random aptamers to form dimeric aptamers. For trimeric or tetrameric aptamers, a small trimeric or tetrameric (i.e., a Holliday junction-like) DNA nanostructure will be engineered to include sequences complementary to the 3′-arm regions of the random aptamers, therefore creating multimeric aptamer fusion through hybridization. In addition, 3 to 5 or 5 to 10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

As used herein, the term “multimeric aptamer” or “multivalent aptamer” refers to an aptamer that comprises multiple monomeric units, wherein each of the monomeric units can be an aptamer on its own. Multivalent aptamers have multivalent binding characteristics. A multimeric aptamer can be a homomultimer or a heteromultimer. The term “homomultimer” refers to a multimeric aptamer that comprises multiple binding units of the same kind, i.e., each unit binds to the same binding site of the same target molecule. The term “heteromultimer” refers to a multimeric aptamer that comprises multiple binding units of different kinds, i.e., each binding unit binds to a different binding site of the same target molecule, or each binding unit binds to a binding site on different target molecule. Thus, a heteromultimer can refer to a multimeric aptamer that binds to one target molecule at different binding sites or a multimeric aptamer that binds to different target molecules. A heteromultimer that binds to different target molecules can also be referred to as a multi-specific multimer.

### 1. Selection of Aptamers Specific to a Target

Aptamers can be artificially generated by a method called systematic evolution of ligands by exponential enrichment (SELEX) (Tuerk and Gold, Systematic evolution of ligands by exponential enrichment: RNA ligands to bacteriophage T4 DNA polymerase. Science, 1990; 249:505-510; and Ellington and Szostak, In vitro selection of RNA molecules that bind specific ligands. Nature, 1990; 346:818-822; the contents of each of which are incorporated herein by reference in their entirety). This method allows the in vitro evolution of nucleic acid molecules with highly specific binding to target molecules. The SELEX method and improvements are further described in, for example, U.S. patent. Nos. 10,546,650, 9,454,642, 8,680,017, 7,964,356, 7,087,735, 6,716,583, 5,817,785, 5,475,096 and 5,270,163; the contents of each of which are incorporated by reference herein in their entirety.

The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric.

The SELEX™ process relies, as a starting point, upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected purpose such as, CpG motifs, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The randomized nucleotides can be produced in a number of ways including chemical synthesis and size selection from randomly cleaved cellular nucleic acids, e.g., phosphodiester-linked nucleotides using solid phase oligonucleotide synthesis techniques well known in the art and solution phase methods such as triester synthesis methods. Typical syntheses carried out on automated DNA synthesis equipment yield 10¹⁴-10¹⁶ individual molecules, a number sufficient for most SELEX™ experiments. The random sequence can be of any length and can comprise ribonucleotides and/or deoxyribonucleotides and can include modified or non-natural nucleotides or nucleotide analogs (see for example U.S. Pat. Nos. 5,958,691 and 5,660,985). Sequence variation in the test nucleic acids can also be introduced or increased by mutagenesis before or during the selection/amplification iterations.

The library of oligonucleotides for aptamer selection may be RNA, DNA or RNA/DNA hybrid. The library of DNA oligonucleotides may be generated by automated chemical synthesis on a DNA synthesizer, while an RNA library of oligonucleotides is typically generated by transcribing a DNA library in vitro using T7 RNA polymerase or modified T7 RNA polymerases. The RNA or DNA library is then mixed with the target (e.g., a biomarker) under conditions favorable for binding and subjected to stepwise iterations of binding, partitioning and amplification, using the same general selection scheme, to achieve the desired criterion of binding affinity and selectivity as defined in the present application.

The binding affinity describes the measure of the strength of the binding or affinity of molecules to each other. Binding affinity of the aptamer herein with respect to targets and other molecules is defined in terms of K_{d}. The dissociation constant can be determined by methods known in the art. It has been observed, however, that for some small oligonucleotides, direct determination of K_{d} is difficult, and can lead to misleadingly high results. Under these circumstances, a competitive binding assay for the target molecule or other candidate substance can be conducted with respect to substances known to bind the target or candidate. The value of the concentration at which 50% inhibition occurs (K,) is, under ideal conditions, equivalent to K_{d}

### 2. Aptamer Modifications

In accordance with the present disclosure, oligonucleotides and aptamers may be further modified to improve their stability. The present disclosure also includes analogs as described herein and/or additional modifications designed to improve one or more characteristics of aptamers such as protection from nuclease digestion. Oligonucleotide modifications contemplated in the present disclosure include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole.

Modifications to generate oligonucleotides which are resistant to nucleases can also include one or more substitute internucleotide linkages, altered sugars, altered bases, or combinations thereof. Such modifications include 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanosine; 3′ and 5′ modifications such as capping; conjugation to a high molecular weight, non-immunogenic compound; conjugation to a lipophilic compound; and phosphate backbone modification.

In some embodiments, the aptamer comprises at least one chemical modification. In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In a preferred embodiment, the high molecular weight, non-immunogenic compound is polyalkylene glycol, and more preferably is polyethylene glycol (PEG). The process of covalent conjugation of PEG to another molecule, normally a drug or therapeutic protein is known as PEGylation. PEGylation is routinely achieved by incubation of a reactive derivative of PEG with the target molecule. The covalent attachment of PEG to a drug or therapeutic protein can mask the agent from the host's immune system, thereby providing reduced immunogenicity and antigenicity, and increase the hydrodynamic size (size in solution) of the agent which prolongs its circulatory time by reducing renal clearance. PEGylation can also provide water solubility to hydrophobic drugs and proteins.

In another preferred embodiment, the 3′ cap is an inverted deoxythymidine cap.

In some embodiments, nucleic acid aptamers are provided in which the P(O)O group is replaced by P(O)S (“thioate”), P(S)S (“dithioate”), P(O)NR2 (“amidate”), P(O)R, P(O)OR′, CO or CH2 (“formacetal”) or 3′-amine (NH—CH2-CH2-), wherein each R or R′ is independently H or substituted or unsubstituted alkyl. Linkage groups can be attached to adjacent nucleotide through a —O—, —N—, or —S— linkage. Not all linkages in the nucleic acid aptamers are required to be identical.

As non-limiting examples, a nucleic acid aptamer can include D-ribose or L-ribose nucleic acid residues and can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, an inverted deoxynucleoside or inverted ribonucleoside, a 2′-deoxy-2′-fluoro-modified nucleoside, a 2′-amino-modified nucleoside, a 2′-alkyl-modified nucleoside, a morpholino nucleoside, a phosphoramidate or a non-natural base comprising nucleoside, or any combination thereof. Alternatively, a nucleic acid aptamer can comprise at least two modified ribonucleosides, at least 3, at least 4, at least 5, at least 6, at least 7, at least 8, at least 9, at least 10, at least 15, at least 20 or more modified ribonucleosides, up to the entire length of the molecule. The modifications need not be the same for each of such a plurality of modified deoxy- or ribonucleosides in a nucleic acid molecule.

Aptamer may comprise modified nucleobase (often referred to in the art simply as “base”) for increasing the affinity and specificity for their target protein. As used herein, “unmodified” or “natural” nucleobases include the purine bases adenine (A) and guanine (G), and the pyrimidine bases thymine (T), cytosine (C) and uracil (U). For example, the modified base may be a pyrimidine modified by a hydrophobic group, such as benzyl group, a naphthyl group, or a pyrrolebenzyl group, at its 5-position. Modified nucleoside may be exemplified as 5-(N-benzylcarboxyamide)-2′-deoxyuridine (called BzdU), 5-(N-naphthylcarboxyamide)-2′-deoxyuridine (called NapdU), 5-(N-4-pyrrolebenzyl carboxyamide)-2′-deoxyuridine (called 4-PBdU), 5-(N-benzylcarboxyamide)-2′-deoxycytidine (called BzdC), 5-(N-naphthylcarboxyamide)-2′-deoxycytidine (called NapdC), 5-(N-4-pyrrolebenzylcarboxyamide)-2′-deoxycytidine (called 4-PBdC), 5-(N-benzylcarboxyamide)-2′-uridine (called BzU), 5-(N-naphthylcarboxyamide)-2′-uridine (called NapU), 5-(N-4-pyrrolebenzylcarboxyamide)-2′-uridine (called 4-PBU). 5-(N-benzylcarboxyamide)-2′-cytidine (called BzC), 5-(N-naphthylcarboxyamide)-2′-cytidine (called NapC), 5-(N-4-pyrrolebenzyl carboxyamide)-2′-cytidine (called 4-PBC), and the like, but not be limited thereto. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, those disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993.

Aptamers may be further modified to provide protection from nuclease and other enzymatic activities. The aptamer sequence can be modified by any suitable methods known in the art. For example, phosphonothioate can be incorporated into the backbone, and 5′-modified pyrimidine can be included in 5′ end of ssDNA for DNA aptamers. For RNA aptamers, modified nucleotides such as substitutions of the 2′-OH groups of the ribose backbone, e.g., with 2′-deoxy-NTP or 2′-fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

In some embodiments, such modified nucleic acid aptamers may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modifications. For example, all purine nucleotides may have one type of modification (or are unmodified), while all pyrimidine nucleotides have another, different type of modification (or are unmodified). In this way, oligonucleotides, or libraries of oligonucleotides are generated using any combination of modifications as disclosed herein.

### 3. A Tamer Ligands Against Basophil Specific Biomarkers

In accordance with the present disclosure, Aptamers and derivatives thereof that specifically bind to a basophil specific biomarker are provided.

In some embodiments, a suitable nucleotide length for an aptamer ranges from about 15 to about 100 nucleotides (nt), and in various other preferred embodiments, 15-30 nt, 20-25 nt, 30-100 nt, 30-60 nt, 25-70 nt, 25-60 nt, 40-60 nt, 25-40 nt, 30-40 nt, any of 18, 19, 20, 21, 22, 23, 24, 25, 26, 27, 28, 29, 30,31, 32, 33, 34, 35, 36, 37, 38, 39 or 40 nt or 40-70 nt in length. In some embodiments, an aptamer may be 41, 42, 43, 44, 45, 46, 47, 48, 49, 50, 51, 52, 53, 54, 55, 56, 57, 58, 59, 60, 61, 62, 63, 64, 65, 66, ,67, 68, 69, or 70 nt in length. In other embodiments, an aptamer may be 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nt in length. However, the sequence can be designed with sufficient flexibility such that it can accommodate interactions of aptamers with two targets at the distances described herein.

In some embodiments, the nucleic acid aptamer comprises one or more regions of double-stranded character. Such double stranded regions may arise from internal self-complementarity or complementarity with a second or further aptamers or oligonucleotide molecule. In some embodiments the double stranded region may be from 4-12, 4-10, 4-8 base pairs in length. In some embodiments the double stranded region may be 5, 6, 7, 8, 9, 10, 11 or 12 base pairs. In some embodiments the double stranded region may form a stein region. Such extended stem regions having double stranded character can serve to stabilize the nucleic acid aptamer. As used herein, the term “double stranded character” means that over any length of two nucleic acid molecules, their sequences form base pairings (standard or nonstandard) of more than 50 percent of the length.

According to certain embodiments of the present disclosure, variants and derivatives of aptamers are provided. The term “derivative” is used synonymously with the term “variant” and refers to a molecule that has been modified or changed in any way relative to a reference or starting aptamer. The nucleic acid sequence of aptamer variants may possess substitutions, deletions, and/or insertions at certain positions within the nucleotide sequence, as compared to a reference or starting sequence. Ordinarily, variants will possess at least about 50% identity (homology) to a reference sequence, and preferably, they will be at least about 80%, more preferably at least about 90% identical (homologous) to a reference sequence.

In some embodiments, variant mimics of aptamers are provided. As used herein, the term “variant mimic” is one which contains one or more nucleic acids which would mimic an activated sequence. The nucleic acid sequences of variant mimics may comprise naturally occurring nucleic acids, or alternatively, non-naturally occurring nucleic acids.

The aptamer-based ligand is labeled with a marker for signal detection. In some embodiments, the ligand is labeled with a fluorescent dye, selecting from, but not limited to, Alex Fluor® fluorophores (such as Alex 514, Alex 532, Alex 546, Alex 555, Alex 568, Alex 594, Alex 610, Alex 633, Alex 635, Alex 647, Alex 660, Alex 680, Alexa 700, Alex 750, Alex 800, Alex 610-R-phycoerythrin (R-PE), Alex 647-R-phycoerythrin (R-PE), Alex 680-R-phycoerythrin (R-PE), and Alex 680-Allophycocyanin (APC)), Allophycocyanin (APC) and its derivatives, Cy fluorophores (e.g., Cy3.5, Cy3-FITC, CY5, CY 5.5, CY7, CY7-APC, CY5.5-APC), Qdots, TRITC, R-PE, Tamara, Rhodamine Red-X, Rox, TruRed, SYPRO red, BODIPY TR, Propidiun iodide and Texas red. In some examples, the fluorescent dye is Alex 647, Cy5, CY3-FITC or Texas red.

As non-limiting examples, aptamer ligands may bind to an identification biomarker of basophils, including but not limited to, CCR3, CD203c, CD123, CD3 and CRTH2. In one preferred embodiment, aptamer ligands are specific to CD203c.

Aptamer ligands of the present disclosure may bind to a basophil activation biomarker, including but not limited to, CD203c, CD63, CD13, CD69, CD107a, CD107b, CD164, CD80, CD86, CD40L, HLA-DR, CD123, CRTH2, CCR3. In one preferred embodiment, aptamer ligands are specific to CD63. Some exemplary aptamers specific to CD63 may include aptamers of SEQ ID No.: 122-183 in US patent application publication No.: US20170044546; and LL4A (Li et al., Molecular Ther. Nucleic Acids, 2019; 18:727-738); the contents of each of which are incorporated herein by reference in their entirety.

In some embodiments, Aptamer ligands bind to the most commonly used basophil activation markers CD63 and CD203c.

### Aptamer-BAT Platform

The present in vitro BAT using nucleic acid ligands (e.g., aptamer ligands) measures the basophil activation induced by an allergen of interest for diagnosing and/or prognosing an individual's allergic reaction to the allergen of interest. This point-of-care aptamer-based BAT platform that can diagnose an allergic reaction within five minutes. In some embodiments, the BAT can diagnose an allergic reaction in under three minutes.

The BAT for measuring the basophil activation induced by an allergen stimulation comprises identifying basophils in the blood sample using a nucleic acid ligand specifically binding to a basophil identification biomarker; and detecting activated basophils with a nucleic acid ligand specific to a biomarker expressed on the cell surface of activated basophils. The basophil activation induced by the allergen of interest is determined by calculating the signal ratio from the identification marker and activation marker.

As a non-limiting example, the basophil identification marker is CD203c, and the nucleic acid ligand is an aptamer specifically binding to CD203c. The activation biomarker is CD63, and the nucleic acid ligand is an aptamer specifically binding to CD63. The basophil activation induced by the allergen of interest is determined by calculating the CD63/CD203c signal ratio.

In accordance with the present disclosure, a method for diagnosing and/or prognosing an individual's allergic reaction to a test substance (e.g., a food allergen) is provided. The method comprises determination of basophil activation induced by the test substance, wherein the basophil activation is measured by the changes of fluorescence signals that indicate expression of two or more biomarkers on the cell surface of basophils; the method comprising the steps of: a) collecting a blood sample from the individual and incubating the blood sample with the test substance to activate the basophils in the blood sample; b) introducing to the blood sample a mixture of nucleic acid ligands comprising an aptamer against an activation biomarker exposed on the cell surface upon activation of basophils, and an aptamer against an identification biomarker expressed on basophils, wherein each aptamer is labeled with a distinct fluorophore; c) capturing the nucleic acid ligands that are not bound to the basophil markers; d) reading the fluorescence signals and measuring the basophil activation; and e) calculating the basophil activation index by correlating the fluorescence signal changes and determining the allergic reaction.

The blood sample may be whole blood from a subject, or isolated peripheral blood mononuclear cells (PBMCs), which include the basophils. PBMCs may be isolated using standard methods known in the art such as density gradient separation and additional negative selection using magnetic particles allows enrichment for basophils (Gibbs, et al., A rapid two-step procedure for the purification of human peripheral blood basophils to near homogeneity. Clin Exp Allergy. 2008; 38(3): 480-485; the contents of which are incorporated herein by reference in their entirety).

In one exemplary embodiment, the blood sample is whole blood. The whole blood sample is collected immediately prior to the performance of the BAT, ow within 4 hours before the BAT. The collected blood sample may also be processed and stored at 4° C. for at least 24 hours before the BAT. The collection of whole blood for BAT is usually done in heparin, and/or other anticoagulants such as ethylenediaminetetraacetic acid (EDTA) or acid citrate dextrose (ACD) to inhibit basophil degranulation. For example, the blood sample may be an anti-coagulated blood sample.

The sample may a human or animal whole blood sample.

The test substance may be an allergen such as a food allergen, a drug allergen, a pathogen allergen or an airborne allergen. In some embodiments, the allergen to induce the basophil activation during the BAT ranges from crude extracts to recombinant or purified single allergen sources.

The nucleic acid ligand (i.e., aptamer and its derivative thereof) may be labeled with a fluorophore selecting from, but not limited to, Alex Fluor @ fluorophores (such as Alex 514, Alex 532, Alex 546, Alex 555, Alex 568, Alex 594, Alex 610, Alex 633, Alex 635, Alex 647, Alex 660, Alex 680, Alexa 700, Alex 750, Alex 800, Alex 610-R-phycoerythrin (R-PE), Alex 647-R-phycoerythrin (R-PE), Alex 680-R-phycoerythrin (R-PE), and Alex 680-Allophycocyanin (APC)), Allophycocyanin (APC) and its derivatives, Cy fluorophores (e.g., Cy3.5, Cy3-FITC, CY5, CY 5.5, CY7, CY7-APC, CY5.5-APC), Qdots, TRITC, R-PE, Tamara, Rhodamine Red-X, Rox, TruRed, SYPRO red, BODIPY TR, Propidium iodide and Texas red.

In some embodiments, the aptamer ligands specific to biomarkers of basophils are conjugated to magnetic beads or the surface of a solid support (e.g., a glass slide or a plastic chip). The stimulated blood sample may be flowed over the solid support. The basophils labeled by the aptamer ligands are captured to the chip and measured.

Nucleic acid molecules can be covalently attached to magnetic particles/beads by methods based on the formation of covalent bonds. Carboxyl and amino groups are the most common reactive groups for attaching ligands to surfaces. In some aspects, a primary amine (—NH2) modifier may be placed to the 5′ end, or 3′ end of a nucleic acid molecule (e.g., SPN), or internally using an amino-C or amino-T modified base. The amino-modified nucleic acid molecules may be attached to magnetic particles using an acylating reagent, for example Carbodiimide (EDC).

In one exemplary embodiment, aptamer ligands or a plurality of aptamer ligand molecules are affixed to a solid support, for example, by attaching the aptamer ligands to the substrate of an array. The aptamer ligands may be disposed with multiple copies of each aptamer in spots of the substrate.

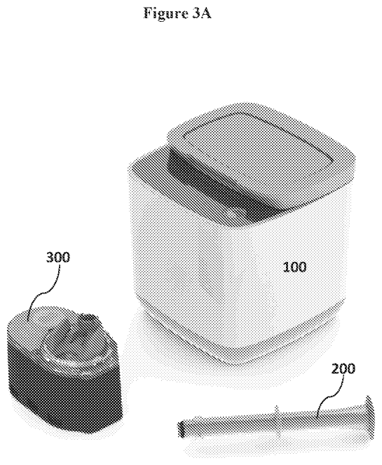

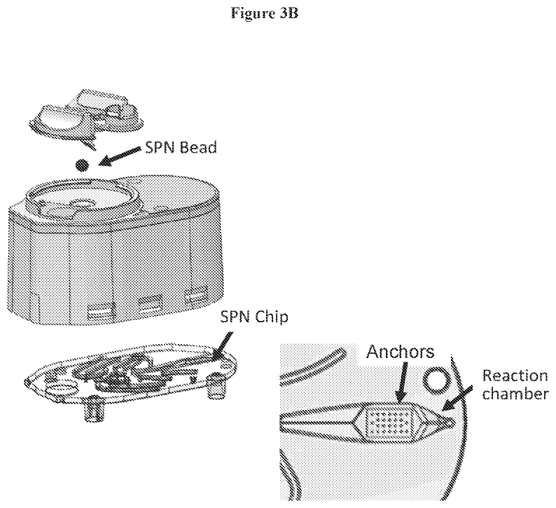

In some embodiments, short sequences complementary to a portion of the sequence of an aptamer ligand conjugated to the surface of the chip are used to capture the aptamer ligands. The short complementary sequences are referred to as “anchors”. The anchors may be covalently immobilized on the surface of the chip (e.g., FIG. 3B).

In some aspects, the complementary sequences (i.e., anchors) may contain about 5 to 20 nucleotide residues, or about 5 to 10 nucleotide residues, or about 10 to 15 nucleotide residues, or about 10-20 nucleotide residues. In particular, it may contain 5, 6, 7, 8, 9, 10, 11, 12, 13, 14, 15, 16, 17, 18, 19, or 20 nucleotide residues. In one embodiment, the complementary sequence contains 5 nucleotide residues. In another embodiment, the complementary sequence contains 10 nucleotide residues. In some aspects, the complementary sequence may be at least 100%, at least 99%, at least 95%, at least 90%, at least 85%, or at least 80% complementary to the aptamer sequence. In another embodiment, the complementary sequence may have additional poly(A) nucleotides. The short complementary sequence can easily detach from the corresponding aptamer ligand.

As used herein, the term “chip” could be understood to be any three-dimensional shape. The substrate may be any types of materials that are suitable for nucleic acid immobilization as discussed above. The materials used as a chip substrate may have the desirable characteristics including optical characteristics, e.g., flatness, transparency, a well-defined optical absorption spectrum, minimal auto-fluorescence, high reflectivity; and chemical characteristics, e.g., surface reactivity that permits covalent linkages.

Aptamer ligands or anchors may be attached to the surface of any solid substrates such as microtiter plates, silicon chips and printed glass surfaces (e.g., epoxy saline-derived glass surfaces). The printed chips may be referred to as microarray chips. Other examples of suitable solid substrates (also called solid supports) may include, but are not limited to, those made of silica or silica-based materials, functionalized glass, modified silicon, inorganic glasses, plastics, resins, polysaccharides, carbon, metals, nylon, natural fibers such as silk, wool and cotton, and polymers. Solid substrates may have any useful form including thin films or membranes, beads, microwell plates, dishes, slides, fibers, woven fibers, shaped polymers, particles, chips, wafers and microparticles. Solid substrates may be porous or non-porous.

In some embodiments, the solid substrate may be a glass or silicon slide. Glass is a readily available and inexpensive support medium that has low intrinsic fluorescence. The surface of the glass or silicon slide may be modified or unmodified, although most attachment protocols involve chemically modifying the glass surface to facilitate attachment of the oligonucleotides. Glass has a relatively homogeneous chemical surface whose properties have been well studied and is amenable to chemical modification using very versatile and well developed silanization chemistry. In certain embodiments, the surface of the glass or silicon slide is unmodified. For example, salinized oligonucleotides can be covalently linked to an unmodified glass surface (Kumar et al., Nucleic Acids Res. 2000; 28(14): e71).

In other embodiments, solid substrates include inorganic materials, e.g., ceramic (such as low temperature cofired ceramic (LTCC)), polymer substrates, composites and paper. Polymers may include elastomers, e.g., polydimethylsiloxane (PDMS; dimethicone), polyester (e.g., thermoset polyester (TPE)); thermoplastic polymers, e.g., polystyrene (PS), polycarbonate (PC), poly-methyl methacrylate (PMMA), and poly-ethylene glycol diacrylate (PEGDA), perfluorinated compounds/polymers (such as perfluoroalkoxy (Teflon PFA), fluorinated ethylenepropylene (Teflon FEP), and polyfluoropolyether diol methacrylate (PFPE-DMA)), and polyurethane (PU); and thermosets, and polyimide and acrylic paper, a flexible cellulose-based material, composite materials, e.g., amorphous material, cyclic olefin polymers (COP), polymers based on cyclic olefin monomers and ethene, such as cyclic olefin copolymer (COC).

In some embodiments, the pre-synthesized aptamer ligands and anchors are conjugated to the solid substrate via generating covalent bonds. Therefore, the nucleic acid molecules are tightly immobilized on the surface, providing high stability of the arrays and reproducibility of the data obtained. In some cases, both nucleic acids and solid surfaces are modified with reactive functional groups to allow chemical reactions to form covalent bonds between the nucleic acid and surface. Commonly used functional groups include but are not limited to carboxyl, phosphate, aldehyde and amino groups. For example, amino groups, can be employed for both the nucleic acid and the surface because of its easy preparation, stable functionality and wide applicability. The solid surface may be modified with amino groups to generate a NH₂-functionalized surface, subsequently subjected to chemical activation by use of homo-bifunctional linkers such as disuccinimidyl glutarate (DSG), phenylene diisothiocyanate (PDC). In other examples, the probe DNA oligonucleotides with carboxyl or phosphate groups at the ends are immobilized on the NH₂-functionalized surface, dehydration reagents such as dicyclohexylcarbodiimide (DCC), 1-ethyl-3-(3-dimethylaminopropyl)carbodiimide (EDC), etc.

For example, the surfaces of glass or silicon can be treated with an amino silane to have a uniform layer of primary amines or epoxides. Nucleic acids modified with an NH₁₂ group can be immobilized onto epoxy silane-derivatized or isothiocyanate coated glass slides. In another example, succinylated nucleic acids can be coupled to aminophenyl- or aminopropyl-derivitized glass slides by peptide bonds. In yet another example, disulfide-modified nucleic acids can be immobilized onto a mercaptosilanized glass support by a thiol/disulfide exchange reactions or through chemical cross linkers. As a non-limiting example, a short poly(A) sequence (e.g., 5 nt) may be added at the end of the aptamer ligand or the complementary sequence. The poly(A) tail then is modified with a thiol group to facilitate the conjugation.

In one exemplary embodiment, the nucleic acids represented may be immobilized to a solid substrate via UV light cross-linking at an exposing wavelength of about 300 nm to 500 nm, preferably at an exposing wavelength of 350 nm. The substrate may comprise a super-hydrophobic polymeric surface.

In some embodiments, the aptamer ligands and/or anchors may be further optimized to increase the conjugation to the solid surface. In some examples, a short linker and spacer sequence may be added to the end(s) of the sequence of the aptamer ligands and/or the anchors. As a non-limiting example, a poly(T) linker sequence may be added to the end of the sequence of the aptamer and/or the anchor (TTTTTTTTTT, SEQ ID NO. 1). The spacer sequence may include but is not limited to, the sequences of SEQ ID NOs.: 2-16.

In other embodiments, aptamer ligands and anchors of the present disclosure may be conjugated to the solid support by in situ oligonucleotide synthesis, i.e., by direct synthesis on a solid surface. A standard 3′ to 5′ phosphonamidite chemical reaction may be used to for oligonucleotide synthesis. Automated synthesizers may be used to synthesize the short complementary anchor sequences.

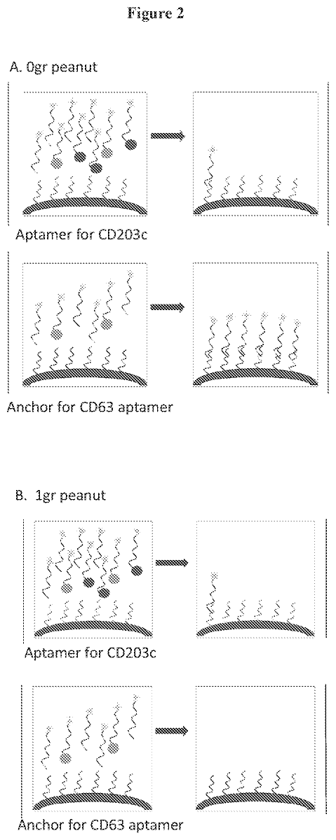

In some embodiments, the anchors complementary to the CD203c aptamer ligand that are conjugated to the chip capture the aptamer ligand against CD203c that are not bound to basophils through hybridization (e.g., free CD203c aptamers) (FIG. 2; top panels of A and B). The fluorescence signals from the free CD203c aptamer ligands defines the baseline intensity of basophils in the blood sample. The anchors complementary to the CD63 aptamer ligand that are conjugated to the chip capture the aptamer ligand against CD63 that are not bound to activated basophils through hybridization (e.g., free CD63 aptamers) (FIG. 2, lower panels of A and B). The fluorescence signals from the free CD63 aptamer ligands correlate the percentage of activated basophils in the blood sample in the presence of an allergen. As illustrated in FIG. 2, when there is no allergen present, basophils are not activated and will be CD63−, anti-CD63 aptamer ligands are captured to the chip through hybridization with the anchors specific to anti-CD63 aptamer ligands (FIG. 2, A. 0 g peanut). The fluorescence signals from anti-CD63 aptamer ligands are measured. However, if an allergen is present, basophils are activated and express CD63 on the cell surface (CD63+), anti-CD63 aptamer ligands bind to CD63 on the cell surface. Only few free anti-CD63 aptamer ligands are captured to the chip through hybridization with the anchors specific to anti-CD63 aptamer ligands (FIG. 2, B. 1 g peanut). The fluorescence signals from anti-CD63 aptamer ligands are measured. The signals from the blood sample without the allergen stimulation have a higher CD63/CD203c ratio, while the signals from the blood sample exposed to the allergen have a lower CD63/CD203c ratio. The signal ratio correlates the concentration of the allergen.

As non-limiting examples, an aptamer ligand that specifically binds to CD63 is used to measure Basophil reactivity in response to allergen exposure. Some exemplary anti-CD63 aptamers may be those developed by Song et al (Song et al., Development of a CD63 aptamer for efficient cancer immunochemistry and immunoaffinity-based exosome isolation. Molecules 2020, 25, 5585; the contents of which are incorporated herein by their entirety). other CD63 specific aptamers may include those selected by Gao et al (Gao et al., a dual signal amplification method for exosome detection based on DNA dendrimer self-assembly; Analyst, 2019, 144, 1995-2002), Yu et al (YU et al., An aptamer-based new method for competitive fluorescence detection of exosomes; Nanoscale, 2019, 11: 15589-15595); the contents of each of which are incorporated by reference herein in their entirety.

As non-limiting examples, an aptamer ligand that specifically binds to CD203c is used to measure Basophil reactivity in response to allergen exposure.

As non-limiting examples, an aptamer ligand that specifically binds to CD123 is used to measure Basophil reactivity in response to allergen exposure. Some exemplary anti-CD123 aptamers may include those developed by Wu et al. (Wu et al., Novel CD123-aptamer-originated targeted drug trains for selectively delivering cytotoxic agent to tumor cells in acute myeloid leukemia theranostics. Drug Deliv., 2017, 24, 1216-1229); and Wang et al (Wang et al., SS30, a novel trioaptamer targeting CD123, inhibits the growth of acute myeloid leukemia cells; Life Science, 2019, 232: 116663); the contents of each of which are incorporated herein by their entirety.

As non-limiting examples, an aptamer ligand that specifically binds to CD193 is used to measure Basophil reactivity in response to allergen exposure.

In some embodiments, the present BAT tests a dose-response to a specific allergen. The dose dependency testing may comprise three to five different allergen concentrations, for instance in 10-fold increments. Accordingly, the results of the BAT form a dose response curve may be measured as basophil reactivity, basophil sensitivity or both. For example, a basophil reactivity to a specific allergen may be measured with the percentage of CD63 positive basophils at a given concentration of the allergen, or with CDₘₐₓ (i.e., the concentration at which maximal basophil activation occurs). Basophil sensitivity may be measured as either EC50 (i.e., the concentration at which 50% of maximal basophil response occurs) or CD-sens (i.e., the inverse of EC50 multiplied by 100 which is calculated from the slope of the dose-response curve). The area under a dose response curve may be used to assess basophil reactivity and sensitivity simultaneously.

Other biomarkers specific to basophil cells may include but are not limited to, CCR3, CD203c, CD123, CD3 and CRTH2 which can identify basophils in the blood sample. Basophil activation markers may include but are not limited to, CD203c, CD63; CD13, CD69, CD107a, CD107b, CD164, CD80, CD86, CD40L, HLA-DR, CD123, CRTH2, CCR3 and other extracellular markers.

Optionally, the present BAT is accompanied with an IgE-dependent (e.g., anti-IgE or anti-FceRI) and IgE-independent (e.g., fMLP or ionomycin) positive controls in the BAT. Furthermore, negative control consisting of stimulation buffer alone should also be included to assess the level of background or spontaneous activation of basophils.

In another aspect, the present BAT may be used for measuring the severity and threshold of an allergic reaction to an allergen, by measuring the changes of fluorescence signals that indicate expression of at least one biomarker on the cell surface of basophils, wherein the expression of the biomarker is measured with a nucleic acid ligand specifically binding to the biomarker. Individuals with more severe reactions show a greater proportion of activated basophils and individuals reacting to trace amounts of the allergen show a greater basophil sensitivity, i.e., their basophils start reacting at lower allergen concentrations.

The present BAT is more accurate than the skin prick test and serum IgE test. Moreover, the present BAT may distinguish individuals that are clinically allergic from those who are tolerant.

The present BAT may have high specificity and sensitivity. The test specificity may range between 60 and 100%, or 65 and 100%, or 70 and 100%, or 75 and 100%, or 80 and 100%, or 90 and 100%. The test sensitivity may range between 75 and 100%, or 80 and 100%, or 90 and 100%.

As a non-limiting example, the assay of the present disclosure may include the steps of

(1) Sample preparation: the collected whole blood sample may be heparinized and diluted with a buffer. The diluted blood sample is introduced into an analytic cartridge which comprises the detection aptamers including signal polynucleotide (SPNs) derived from the aptamers, which target to biomarkers specific to basophils and activated basophils.

(2) Labeling and capturing basophils: the SPN bound basophil samples are brought into contact with an array chip that is printed with multiple anchors that are short DNA sequences complementary to the sequences of the SPN molecules. The anchors include test anchor sequences that are complementary to the active domain of the SPN targeting to a biomarker of basophil and competes with binding to the target analyte (allergen), and control ancho sequences that are complementary to the non-active domain of the SPN targeting to a biomarker of basophil, regardless of whether the analyte is bound to the SPN molecule.

(3) Signal detection and process: the signals from the test anchors and control anchors for each biomarker are calculated. An algorithm is used to analyze the intensities of signals from the test and control anchors for each biomarker. All signals are calibrated based on the control anchors. The signal parameters include the presence of basophils and activated basophiles in the sample.

In some embodiments, the SPN molecules that target to CD123 and CD63 are used in combination in a diagnostic assay. The presence of basophils in the sample may be determined by the ratio between the test and control anchors of the SPN molecules targeting to CD123. The presence of activated basophils in the sample may be determined by the ratio between the test and control anchors of the SPN molecules targeting to CD63. The ratio between the presence of basophils and activated basophils in the sample is calculated and used to indicate the allergic reaction.

In other embodiments, the SPN molecules that target to CD203c and CD193 are used in a diagnostic assay.

As disclosed herein, the present assay for detecting basophil response to allergens is a comparative system enabling a clear output based on a calibrated comparison of the patient's cells.

### Applications-Allergic Reactions

### BAT in Diagnosing Food Allergies

The present BAT can be used for diagnosing, prognosing and monitoring food allergies, the present BAT is of use for measuring the severity of allergic reactions to food allergies.

Examples of foods are eggs, milk, meat, fishes, crustacea and mollusks, cereals, legumes and nuts, fruits, vegetables, beer yeast, and gelatin; more particularly, egg white and egg yolk of the eggs, milk and cheese of the milk, pork, beef, chicken and mutton of the meat, mackerel, horse mackerel, sardine, tuna, salmon, codfish, flatfish and salmon caviar of the fishes, crab, shrimp, blue mussel, squid, octopus, lobster and abalone of the crustacea and mollusks, wheat, rice, buckwheat, rye, barley, oat, corn, millet, foxtail millet and barnyard grass of the cereals, soybean, peanut, cacao, pea, kidney bean, hazelnut, Brazil nut, almond, coconut and walnut of the legumes and nuts, apple, banana, orange, peach, kiwi, strawberry, melon, avocado, grapefruit, mango, pear, sesame and mustard of the fruits, tomato, carrot, potato, spinach, onion, garlic, bamboo shoot, pumpkin, sweet potato, celery, parsley, yam and Matsutake mushroom of the vegetables, the foods containing them, and the ingredients thereof.

In some embodiments, the present in vitro BAT is used for diagnosing, prognosing and monitoring an individual's allergic reactions to common food allergens including, but not limited to peanut, tree nuts, egg, wheat, milk, soy, fish and shellfish.

Examples of allergenic proteins associated with food include, but are not limited to, Brine shrimp (Art fr 5), Crab (Cha f 1), North Sea Shrimp (Cra c 1, Cra c 2, Cra c 4, Cra c 5, Cra c 6, Cra c 8), American lobster (Hom a 1, Hom a 3, Hom a 6), white shrimp (Lit v 1, Lit v 2, Lit v 3, Lit v4), giant freshwater prawn (Mac r 1), shrimp (Met e 1, Pen a 1, Pen i 1), northern shrimp (Pan b 1), spiny lobster (Pan s 1), black tiger shrimp (Pen m 1. Pen m 2. Pen m 3. Pen m 4, Pen m 6), narrow-clawed crayfish (Pon i 4, Pon i 7), blue swimmer crab (Por p 1), domestic cattle (Bos d 4, Bos d 5, Bos d 6, Bos d 7, Bos d 8, Bos d 9, Bos d 10, Bos d 11, Bos d 12), Atlantic herring (Clu h 1), common carp (Cyp c 1), Baltic cod (Gad c 1), Atlantic cod (Gad m 1, Gad m 2, Gad m 3), cod (Gad c 1), chicken (Gal d 1, Gal d 2, Gal d 3, Gal d 4, Gal d 5), Barramunda (Lat c 1), Lepidorhombus whiffiagonis (Lep w 1), chum salmon (Onc k 5), Atlantic salmon (Sal s 1, Sal s 2, Sal s 3) rainbow trout (One m 1), Mozambique tilapia (Ore m 4), edible frog (Ran e 1, Ran e 2), pacific pilchard (Sar sa 1), ocean perch (Seb m 1), yellowfin tuna (Thu a 1, Thu a 2, Thu a 3), swordfish (Xip g 1), abalone (Hal m 1), brown garden snail (Hel as 1), Squid (Tod p 1), pineapple (Ana c 1, Ana c 2), asparagus (Aspa o 1), barley (Hor v 12, Hor v 15, Hor v 16, Hor v 17, Hor v 20, Hor v 21), banana (Mus a 1, Mus a 2, Mus a 3, Mus a 4, Mus a 5), banana (Musxp1), rice (Ory s 12), rye (See c 20), wheat (Tri a 12, Tri a 14, Tri a 18, Tri a 19, Tri a 25, Tri a 26, Tri a 36, Tri a 37), maize (corn) (Zea in 14, Zea in 25), kiwi fruit (Act c1, Act c 2, Act c 5, Act c 8, Act c 10, Act d 1, Act d 2, Act d 3, Act d 4, Act d 5, Act d 6, Act d 7, Act d 8, Act d 9, Act d 10, Act d 11), cashew (Ana o 1, Ana o 2, Ana o 3), celery (Api g 1, Api g 2, Api g 3, Api g 4, Api g 5, Api g 6), peanut (Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13), brazil nut (Ber e 1, Ber e 2), oriental mustard (Braj 1), rapeseed (Bra n 1), cabbage (Bra o 3), turnip (Bra r 1, Bra r 2), bell pepper (Cap a 1w, Cap a 2), pecan (Car i 1, Car i 4), chestnut (Cas s 1, Cas s 5, Cas s 8, Cas s 9), lemon (Cit I 3), tangerine (Cit r 3), sweet orange (Cit s 1, Cit s 2, Cit s 3), Hazel (Cor a 1, Cor a 2, Cor a 8, Cor a 9, Cor a 11, Cor a 12, Cor a 13, Cor a 14), muskmelon (Cuc in 1, Cuc m 2, Cuc in 3), carrot (Dau c 1, Dau c 4, Dau c 5), common buckwheat (Fag e 2, Fag e 3), tartarian buckwheat (Fag t 2), strawberry (Fra a 1, Fra a 3, Fra a 4), soybean (Gly in 1, Gly in 2, Gly in 3, Gly in 4, Gly in 5, Gly in 6, Gly in 7, Gly in 8), sunflower (Hel a1, Hel a 2, Hel a 3), black walnut (Jug n 1, Jug n 2), English walnut (Jug r 1, Jug r 2, Jug r 3, Jug r 4), Cultivated lettuce (Lac s 1), Lentil (Len c 1, Len c 2, Len c 3), litchi (Lit c 1), narrow-leaved blue lupin (Lup an 1), apple (Mal d 1, Mal d 2, Mal d 3, Mal d 4), Cassava (Man e 5), mulberry (Mor n 3), avocado (Pers a 1), green bean (Pha v 3), pistachio (Pis v 1, Pis v 2, Pis v 3, Pis v 4, Pis v 5), pea (Pis s 1, Pis s 2), apricot (Pru ar 1, Pru am 3), sweet cherry (Pru av 1, Pru av 2, Pru av 3, Pru av 4), European plum (Pru d 3), almond (Pru du 3, Pru du 4, Pru du 5, Pru du 6), peach (Pru p 1, Pru p 2, Pru p 3, Pru p 4, Pru p 7), pomegranate (Pun g 1), pear (Pyr c 1, Pyr c 3, Pyr c 4, Pyr c 5), castor bean (Ric c 1), red raspberry (Rub i 1, Rub i 3), Sesame (Ses i 1, Ses i 2, Ses i 3, Ses i 4, Ses i 5, Ses i 6, Ses i 7), yellow mustard (Sin a 1, Sin a 2, Sin a 3, Sin a 4), tomato (Sola I 1, Sola I 2, Sola I 3, Sola I 4), potato (Sola t 1, Sola t 2, Sola t 3, Sola t 4), Mung bean (Vig r 1, Vig r 2, Vig r 3, Vig r 4, Vig r 5, Vig r 6), grape (Vit v 1). Chinese date (Ziz m 1), Anacardium occidentale (Ana o 1.0101, Ana o 1.0102). Apium graveolens (Api g 1.0101, Api g 1.0201), Daucus carota (Dau c1.0101, Dau c1.0102, Dau c1.0103, Dau c1.0104, Dau c1.0105, Dau c1.0201), Citrus sinensis (Cit s3.0101, Cit s3.0102), Glycine max (Gly m1.0101, Gly m1.0102, Gly m3.0101, Gly m3.0102), Lens culinaris (Len c1.0101, Len c1.0102, Len c1.0103), Pisumn sativum (Pis s1.0101, Pis s1.0102), Lycopersicon sativum (Lyc e2.0101, Lyc e2.0102), Fragaria ananassa (Fra a3.0101, Fra a3.0102, Fra a3.0201, Fra a3.0202, Fra a3.0203, Fra a3.0204, Fra a3.0301), Malus domestica (Mal d1.0101, Mal d1.0102, Mal d1.0103, Mal d1.0104, Mal d1.0105, Mal d1.0106, Mal d1.0107, Mal d1.0108, Mal d1.0109, Mal d1.0201, Mal d1.0202, Mal d1.0203, Mal d1.0204, Mal d1.0205, Mal d1.0206, Mal d1.0207, Mal d1.0208, Mal d1.0301, Mal d1.0302, Mal d1.0303, Mal d1.0304, Mal d1.0401, Mal d1.0402, Mal d1.0403, Mal d3.0101w, Mal d3.0102w, Mal d3.0201w, Mal d3.0202w, Mal d3.0203w, Mal d4.0101, Mal d4.0102, Mal d4.0201, Mal d4.0202, Mal d4.0301, Mal d4.0302), Prunus avium (Pru av1.0101, Pru av1.0201, Pru av1.0202, Pru av1.0203), and Prunus persica (Pru p4.0101, Pru p4.0201); and any variants thereof. The names of allergens associated with food are systematically named and listed according to IUIS Allergen Nomenclature Sub-Committee (see, International Union of Immunological Societies Allergen Nomenclature Sub-Committee, List of isoallergens and variants.)

### BAT in Diagnosing Drug Allergies

Drug allergy is an allergic reaction to a drug, a type of drug hypersensitivity reaction (IDHR), in which there is a specific immune response to a drug mediated by immunoglobulins (IgE) and/or T cells. The present in vitro BAT is applied in the diagnosis of IDHR. Adverse allergic reactions to drugs are diverse and make the diagnosis more difficult. This challenge posts a request for improved methods for testing. For example, skin testing to drugs, particularly intradermal testing, incurs a significant risk of systemic reactions, including anaphylaxis. Furthermore, sIgE testing is not possible to both the native drug and all its metabolites. Drug provocation tests (DPTs) to certain drugs are impractical or unethical particularly in the context of anaphylaxis under general anesthesia. The present in vitro BAT, however, provides a cheaper and safer alternative diagnostic tool to other tests.

The present in vitro BAT may be used for diagnosing an allergic response to a range of drugs including but not limited to, betalactams, quinolones (ciprofloxacin, moxifloxacin and levofloxacin), NMBAs (neuromuscular blocking agents) and antibiotics (e.g., β-lactams and fluoroquinolones).

In some embodiments, the present in vitro BAT can serve as a biomarker for anaphylaxis following drug desensitization. Drug desensitization is imperative for allergic patients requiring full therapeutic doses of lifesaving medication. In this context, the BAT can identify patients allergic to medicines with high risk of adverse reactions during drug desensitization.

### BAT in Diagnosing Other Allergies

The present in vitro BAT may be used in the diagnosis of airborne allergy, including but not limited to birch pollen and grass pollen. The BAT may also be used to diagnose allergic reactions to other inhaled allergens that are caused by IgE-mediated immune responses.

Exemplary airborne particulates/allergens and other environmental allergens include, but are not limited to, allergens may be obtained from plants (e.g. weeds, grasses, trees, pollens), animals (e.g., allergens found in the dander, urine, saliva, blood or other bodily fluid of mammals such as cat, dog, cow, pig, sheep, horse, rabbit, rat, guinea pig, mouse and gerbil), fungi/mold, insects (e.g., stinging insects such as bee, wasp, and hornet and chirnomidae (non-biting midges), as well as other insects such as the housefly, fruit fly, sheep blow fly, screw worm fly, grain weevil, silkworm, honeybee, non-biting midge larvae, bee moth larvae, mealworm, cockroach and larvae of Tenibrio molitor beetle; spiders and mites such as the house dust mite), rubbers (e.g. latex), metals, chemicals (e.g. drugs, protein detergent additives) and autoallergens and human autoallergens (e.g. Hom s 1, Hom s 2, Hom s 3, Hom s 4, Hom s 5) (see, Allergen Nomenclature: International Union of Immunological Societies Allergen Nomenclature Sub-Committee, List of allergens and Allergen Nomenclature: International Union of Immunological Societies Allergen Nomenclature Sub-Committee, List of isoallergens and variants).

The present in vitro BAT may be used in the diagnosis of allergies to pathogens such as bacteria, fungi and mold.

The present in vitro BAT may be used in the diagnosis of venom allergy, including but not limited to, hymenoptera venom allergy (Korosec et al., Clinical routine utility of basophil activation testing for diagnosis of Hymenoptera-allergic patients with emphasis on individuals with negative venom-specific IgE antibodies. Int Arch Allergy Immunol. 2013; 161(4): 363-368; the contents of which are incorporated herein by reference in their entirety), bee and wasp venoms. Another application of BAT in venom allergy is monitoring of immunotherapy treatment. The BAT can be performed using native extracts but also with recombinant venom proteins.

The present in vitro BAT may be used in the diagnosis of respiratory allergy. Allergic reactions to inhaled allergens are heterogeneous and can be complex because of diverse allergens that a subject is naturally exposed to. Some patients who suffer from local allergic rhinitis, may have undetectable levels of sIgE and negative skin tests, making sIgE quantification and SPT test unable to differentiate between allergic and non-allergic rhinitis. The BAT assay may be used to test unique aspects of allergic rhinitis and allergic asthma with basophil sensitivity and specificity.

### Other Applications

In some embodiments, the present in vitro BAT may be used as a tool in the understanding of the mechanisms of some allergic diseases, e.g., chronic urticaria, atopic dermatitis. The present BAT can be used to characterize the surface proteins expressed by basophils and how these modulate the response to allergen stimulation or challenge. As non-limiting examples, the present BAT assays may be used to quantify FcεRI expression, as well as the expression of other Fcgamma receptors (CD16, CD32, CD64) in the steady state and also in response to activation or challenge.

In some embodiments, the present in vitro BAT may be used to compare therapeutics under experimental conditions, such as to test the effectiveness of IgE inhibitors. As non-limiting examples, the present BAT may be used to measure receptor-bound antibodies on the surface of effector cells. The changes in activated basophils, e.g., reduction in basophil numbers, may indicate efficacy of anti-allergy treatments.

In some embodiments, the present in vitro BAT can test basophil response to allergen stimulation, e.g., the characteristics of allergen specific IgE such as concentration, specificity, clonality and affinity for allergen. As a non-limiting example, the present BAT assay can test and compare basophil responses to different allergenic proteins and epitopes. The improved understanding of allergic peptides and epitopes which elicit allergic responses are key for both improved diagnostic tests and potential novel treatments.

In some embodiments, the present in vitro BAT can test basophil activation independent of IgE induction.

In some embodiments, the present in vitro BAT can determine basophil response to allergens may be used for monitoring patients submitted to allergen specific immunotherapy (AIT) and other immunomodulatory treatments such as anti-IgE treatments. For example, a decrease in basophil sensitivity has also been reported following treatment with omalizumab across a range of allergies including allergy to peanut, cat and Aspergillus (Gernez et al., Basophil CD203c levels are increased at baseline and can be used to monitor omalizumab treatment in subjects with nut allergy. Int Arch Allergy Immunol. 2011; 154(4): 318-327; Johansson et al., The size of the disease relevant IgE antibody fraction in relation to ‘total-IgE’ predicts the efficacy of anti-IgE (Xolair) treatment. Allergy. 2009; 64(10): 1472-1477; and Voskamp et al., Clinical efficacy and immunologic effects of omalizumab in allergic bronchopulmonary aspergillosis. J Allergy Clin Immunol Pract. 2015; 3(2): 192-199; the contents of each of which are incorporated herein by reference in their entirety).

### Kits

In accordance with the present disclosure, aptamer ligands, anchors complementary to the aptamer ligands, beads and solid substrates conjugated with anchors or aptamer ligands are packaged to form kits for measuring allergic reactions. The compositions may be combined with other ingredients or reagents or prepared as components of kits or other retail products for commercial sale or distribution.

The kit will contain agents of the present disclosure, along with instructions regarding administration and/or use of the kit. In some embodiments, the kit further comprises sample collectors for collecting blood samples.

### EXAMPLES

### Example 1: Sample Preparation and Basophil Label

The whole blood sample from a patient is heparinized and diluted with buffer. The sample diluents are introduced into four different analytic containers (e.g., the disposable pod 300 in FIGS. 3A and 3B), among which one container is served as a control. The blood sample diluents in the other tree containers are then further exposed to the peanut allergen at different concentrations (i.e., increased levels of the peanut allergen). The amount of allergen depends on the patient's history. The mixtures are then incubated for approximate 30 minutes.

In each analytical container, the detection agents, i.e., the signaling polynucleotides (SPNs) specific to the activated basophil markers CD123 and CD63, are preloaded in the container. The blood samples pre-stimulated by the peanut allergen are mixed and exposed to the SPNs targeting CD123 and CD63 which bind specifically to basophils. The mixtures are introduced to the capture chip (i.e., the anchor chip in the reaction chamber of the disposable pod 300 of FIGS. 3A and 3B) which includes an array with multiple printed anchors that comprise short sequences complementary to the active domain of the SPN molecules of CD63 and CD123, and control anchors that comprise short sequences complementary to the non-active domain of the SPN molecules of CD63 and CD123.

Multiple capture images of the array chips are collected as the sample is introduced to the reaction chamber to document the reaction rate, after inserting the analytic container to an instrument (e.g., the detection device 100 in FIG. 3A). The images are analyzed and processed for signal detection. An algorithm is used to analyze the fluorescence intensities from the test and control anchors for both CD123 and CD63. Several parameters, including the presence of basophils in the sample (by the ratio between the test and control anchors for the SPNs targeting to CD123) and the presence of activated basophiles in the sample (by the ratio between the test and control anchors for the SPNs targeting to CD63, are calculated. All signals are further calibrated based on the control anchors.

### Example 2: Signal Analysis of Aptamer-Based BAT Assay

Aptamers that bind CD123 and CD63 are used to test BAT signal analysis using the detection system of FIGS. 3A and 3B.

TABLE 1
Aptamer sequences

| | | SEQ ID |
|---|---|---|
| Aptamer | Sequence (5′-3′) | NO. |
| CD63 | TAACACGACAGACGTTCGGAGGTCGAACCCTGACA | 17 |
| | GCGTGGGC | |
| CD63 | TAACCACCCCACCTCGCTCCCGTGACACTAATGCT | 18 |
| | AATTCCAA | |
| CD123 | TGCGTGTGTAGTGTGTCTGGGCTACATCGATGAGC | 19 |
| | TGCCTAGGGTCCCTCTTAGGGATTTGGGCGG | |
| CD123 | TGCGTGTGTACTGTGTCTGGTCCCGTAGCTACTAG | 20 |
| | CGAACTCCCTGCCTCTTAGGGATTTGGGCGG | |

The aptamers specific to CD63 (SEQ ID Nos.: 17 and 18) are discussed in Zhong et al. (Zhong et al., Development of a CD63 aptamer for efficient cancer immunochemistry and immunoaffinity-based exosome isolation; Molecules, 2020, 25: 5585-5601). The aptamers specific to CD123 (SEQ ID NOs.: 19 and 20) are discussed in Wu et al., (Wu et al., Novel CD123-aptamer-originated targeted drug trains for selectively delivering cytotoxic agent to tumor cells in acute myeloid leukemia theranostics; Drug Deliver, 2017, 24(1): 1216-1229).

Three peanut samples at different concentrations (1 mg, 10 mg and 100 mg) are tested and compared. A sample free of peanut (0 mg peanut) is used as control.

TABLE 2
Calibrated signals at different allergen (e.g., peanut) concentrations

| | | Calibrated | Calibrated | | | | | |
|---|---|---|---|---|---|---|---|---|
| | | CD123+ | CD123+ | CD63 | CD63 | | Total | |
| | | test anchor | control anchor | test | control | Total | activated | % active |
| Peanut | | intensity | intensity | anchor | anchor | basophils | basophils | basophils |
| 0 | mg | 1/1 | 3/3 | 3/3 | 3/3 | 1/3 | 3/3 | 3/1 |
| 1 | mg | 1/1 | 3/3 | 2/3 | 3/3 | 1/3 | 3/2 | 9/2 |
| 10 | mg | 1/1 | 3/3 | 1/3 | 3/3 | 1/3 | 3/2 | 9/1 |
| 100 | mg | 1/1 | 3/3 | 0/3 | 3/3 | 1/3 | 3/0 | 9/0 |

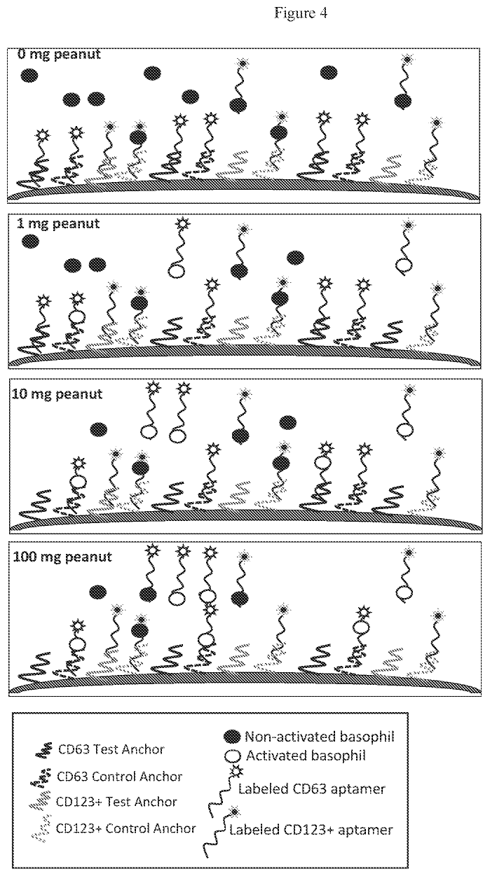

In this exemplary assay, 4 disposable pods are used to calculate the BAT assay. In each test pod, a SPN specific to CD123 and a SPN specific to CD63 are used to capture and label CD123positive basophils and activated CD63 positive basophils in response to the allergen at different concentrations. Each pod contains a chip coated with anchors specific to CD123 SPN and CD63 SPN, respectively. The detection chip is also coated with control anchor sequences for CD123 and CD63 respectively (as FIG. 3B and FIG. 4).

Pod #0: No allergen is added to this pod. Non-activated basophils bind CD123+ SPN. The intensity will be bright on the CD123+ Control anchor and dim on the CD123+ Test anchor. As there are no activated Basophils, the intensity of the CD63 test and control anchors is bright. The intensity of the CD63 & CD123+ anchors will be used to calibrate CD63 signaling in pods with addition of allergen (FIG. 4).

Pod #1: 1 mg peanut allergen is added to the pod. Non-activated basophils bind CD123+ SPN. The intensity will be bright on the CD123+ Control anchor and dim on the CD123+ Test anchor. Some basophils are activated in response to allergen; therefore, the intensity of the CD63 test anchor will decrease while the intensity of the CD63 control anchor will be bright. The intensity of the CD63 & CD123+ anchors will be calibrated to CD63 and CD123+ signaling in pod #0 with addition of allergen.

Pod #2: 10 mg peanut allergen is added to the pod. Non-activated basophils bind CD123+ SPN; therefore, the intensity will be bright on the CD123+ control anchor and dim on the CD123+ test anchor. More Basophils are activated in response to increased amount of allergen; therefore, the intensity of the CD63 test anchor will decrease while the intensity of the control anchor will be bright. The intensity of the CD63 & CD123+ anchors will be calibrated to CD63 and CD123+ signaling in pod #0 with addition of allergen.

Pod #3: 100 mg peanut allergen is added to the pod. Non-activated basophils bind CD123+ SPN; therefore, the intensity will be bright on the CD123+ control anchor and dim on the CD123+ Test anchor. Increased numbers of basophils are activated in response to increased amount of allergen; therefore, the intensity of the CD63 test anchor will decrease while the intensity of the control anchor will be bright. The intensity of the CD63 & CD123+ anchors will be calibrated to CD63 and CD123+ signaling in pod #0 with addition of allergen. Signal calibration

Step 1: Four signals from CD123, CD63 with and without allergen are calibrated.

$$( \frac{CD 123 + Test Anchor Intensity With Allergen}{CD 123 + Test Anchor Intensity With out Allergen} ) = Calibrated CD 123 + Test Anchor Intensity$$

$$( \frac{CD 123 + Control Anchor Intensity With Allergen}{CD 123 + Control Anchor Intensity With out Allergen} ) = Calibrated CD 123 + Control Anchor Intensity$$

$$( \frac{CD 63 Test Anchor Intensity With Allergen}{CD 63 Test Anchor Intensity With out Allergen} ) = Calibrated CD 63 Test Anchor Intensity$$

$$( \frac{CD 63 Control Anchor Intensity With Allergen}{CD 63 Control Anchor Intensity With out Allergen} ) = Calibrated CD 63 Control Anchor Intensity$$

Step 2: The 4 signals from step 1 are used to calculate the total basophils and total activated basophils in the test sample.

$$( \frac{Calibrated CD 123 + Test Anchor Intensity}{Calibrated CD 123 + Control Anchor Intensity} ) = Total Basophils in Sample$$

$$( \frac{Calibrated CD 63 Test Anchor Intensity}{Calibrated CD 63 Control Anchor Intensity} ) = Total Activated Basophils in Sample$$

Step 3: Calculate the percentage of activated basophils in the test sample.

$$( \frac{Total Activated Basophils in sample}{Total Basophils in sample} ) = \% Activated Basophils in Sample$$

The ratio calculated between total basophils and activated basophils is used as a function of allergen concentration. All signals are calibrated based on the control anchors.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

A number of possible alternative features are introduced during the course of this description. It is to be understood that, according to the knowledge and judgment of persons skilled in the art, such alternative features may be substituted in various combinations to arrive at different embodiments of the present disclosure.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects. While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method for diagnosing or prognosing an individual's allergic reaction to a test substance comprising determination of the basophil activation induced by the test substance; the method comprising the steps of:
a) collecting a blood sample from the individual and incubating the blood sample with the test substance to activate the basophils in the blood sample;
b) introducing to the blood sample a mixture of nucleic acid ligands comprising an aptamer against an activation biomarker exposed on the cell surface upon activation of basophils, and an aptamer against an identification biomarker expressed on basophils, wherein each aptamer is labeled with a distinct fluorophore;
c) capturing the nucleic acid ligands that are not bound to the basophil biomarkers;
d) reading the fluorescence signals and measuring the basophil activation; and
e) calculating the basophil activation index by correlating the fluorescence signal changes and determining the allergic reaction.

2. The method of claim 1 wherein the method further comprises incubating the blood sample from the individual with a positive control and a negative control, wherein the negative control is achieved by determining the basophil activation in the absence of the test substance.

3. The method of claim 2 wherein the nucleic acid ligands that are not bound to the basophil markers are captured to a chip that is coated with short sequences complementary to a portion of the sequences of the aptamer ligands.

4. The method of claim 3 wherein the fluorescence signals from the aptamer ligands against the basophil activation biomarker that are captured on the chip and the fluorescence signals from the aptamer ligands against the basophil identification biomarker that are captured on the chip are read and quantitated.

5-6. (canceled)

7. The method of claim 4 wherein the basophil activation biomarkers are selected from the group consisting of CD63, CD13, CD107a, CD107b, CD164 and CD69, and wherein the identification biomarkers are selected from the group consisting of CD203c, CCR3, IgE, CD123, CD193 and CRTH2.

8. The method of claim 1 wherein the test substance is an allergen.

9. The method of claim 8 wherein the allergen is selected from the group comprising a food allergen, an airborne allergen, a drug allergen, an environmental allergen, or a pathogen allergen.

10. The method of claim 9 wherein the food allergen is selected from the group consisting of peanut, tree nuts, milk, egg, soy, wheat, fish or shellfish.

11. A method for determining the severity and threshold of an individual's allergic reaction to an allergen of interest, by measuring the fluorescence signals that indicate the percentage of activated basophils upon exposure to the allergen of interest; the method comprising the steps of:
a) incubating a blood sample with the allergen of interest to activate the basophils in the blood sample;
b) introducing to the blood sample a mixture of nucleic acid ligands comprising an aptamer against an activation biomarker exposed on the cell surface upon activation of basophils, and an aptamer against an identification biomarker expressed on basophils, wherein each aptamer is labeled with a distinct fluorophore;
c) capturing the nucleic acid ligands that are not bound to the basophil markers;
d) reading the fluorescence signals and measuring the basophil activation; and
e) calculating the basophil activation index by correlating the fluorescence signal changes.

12. The method of claim 11 wherein the aptamer ligands that are not bound to the basophil markers are captured to a chip that is coated with short sequences complementary to a portion of the sequences of the aptamer ligands.

13. The method of claim 12 wherein the fluorescence signals from the nucleic acid ligands against the basophil activation biomarker that are captured on the chip and the fluorescence signals from the nucleic acid ligands against the basophil identification biomarker that are captured on the chip are read and quantitated.

14. The method of claim 13 wherein the blood sample is incubated with an allergen of interest in at least three different concentrations; and wherein the basophil activation index is determined for each concentration, thereby determining the severity and threshold of the allergic reaction to the allergen of interest.

15. The method of claim 14 wherein the activation biomarker is CD63 and the identification biomarker is CD203c or CD123.

16. A method for determining basophil activation induced by a test substance comprising measuring the fluorescence signals that indicate the percentage of activated basophils upon exposure to the test substance; the method comprising the steps of:
a). incubating a blood sample with the test substance to activate the basophils in the blood sample;
b) introducing to the blood sample a mixture of nucleic acid ligands comprising an aptamer against an activation biomarker exposed on the cell surface upon activation of basophils, and an aptamer against an identification biomarker expressed on basophils, wherein each aptamer is labeled with a distinct fluorophore;
c) capturing the nucleic acid ligands that are not bound to the basophil markers;
d) reading the fluorescence signals and measuring the basophil activation; and
e) calculating the basophil activation index by correlating the fluorescence signal changes.

17. The method of claim 16 wherein the aptamer ligands that are not bound to the basophil markers are captured to a chip that is coated with short sequences complementary to a portion of the sequences of the aptamer ligands.

18. The method of claim 17 wherein the fluorescence signals from the aptamer ligands against the basophil activation biomarker that are captured on the chip and the fluorescence signals from the aptamer ligands against the basophil identification biomarker that are captured on the chip are read and quantitated.

19. The method of claim 18 wherein the activation biomarker is CD63 and the identification biomarker is CD203c.

20. The method of claim 19 wherein the test substance is an allergen.

21. The method of claim 20 wherein the allergen is selected from the group comprising a food allergen, a drug, an airborne allergen, an environmental allergen, or a pathogen allergen.

22. The method of claim 21 wherein the food allergen is selected from the group consisting of peanut, tree nuts, milk, egg, soy, wheat, fish or shellfish.

23. A method for diagnosing or prognosing an allergic reaction of a subject to a test substance comprising (i) measuring at least one identification biomarker and at least one activation biomarker on the surface of basophils, wherein the basophils are identified using an aptamer ligand that binds to said identification biomarker on the surface of basophils and wherein the activated basophils are captured using an aptamer ligand that binds to said activation biomarker on the surface of activated basophils; and (ii) detecting basophils using anchors that are complementary to said aptamer ligands.

24. The method of claim 23 further comprising assessing the basophil activation state before stimulation with the test substance.

25. The method of claim 23, wherein the aptamer ligands that are not bound to the basophil activation biomarker or identification biomarker are captured to a chip that is coated with the anchor sequences that recognize the aptamer ligands.

26. The method of claim 25, wherein the chip further comprises a control anchor sequence of the aptamer ligand of the activation biomarker and a control anchor sequence of the aptamer ligand of the identification biomarker.

27. The method of claim 24, wherein the identification biomarker on the surface of basophils is selected from the group consisting of CCR3, CD203c, CD193, CD123, IgE and CRTH2; and wherein the activation biomarker on the surface of activated basophils is selected from the group consisting of CD63, CD13, CD107a, CD107b, CD164, and CD69.

28. (canceled)
